(19) United States
(12) Patent Application Publication
(10) Pub. No.: US 2008/0242646 A1
(43) Pub. Date: Oct. 2, 2008

(54) Split dose corticosteroid therapy

(76) Inventors: Jan N. Lessem, Natick, MA (US); Alexis Borisy, Arlington, MA (US); Daniel S. Grau, Arlington, MA (US); M. James Nichols, Boston, MA (US)

Correspondence Address: CLARK & ELBING LLP, 101 FEDERAL STREET, BOSTON, MA, 02110, US

(21) Appl. No.: 12/079,098

(22) Filed: Mar. 25, 2008

Related U.S. Application Data
(60) Provisional application No. 60/920,011, filed on Mar. 26, 2007.

(51) Int. Cl. A61K 31/573

(52) U.S. Cl. 514/170; 514/179

(57) ABSTRACT

The invention features methods, compositions, and kits for split dose corticosteroid therapy for the treatment musculoskeletal disorders, periodontal disease, and immunoinflammatory disorders.

### CROSS-REFERENCE TO RELATED APPLICATIONS

This application claims benefit from U.S. Application No. 60/920,011, filed Mar. 26, 2007, which is incorporated herein by reference.

### BACKGROUND OF THE INVENTION

Musculoskeletal disorders such as arthritis are among the most frequent causes of physical disability among older adults. The three most common types of arthritis are osteoarthritis (OA), rheumatoid arthritis (RA), and gout. Osteoarthritis is the most common joint disease, with radiological evidence of its existence found in 50% of the population.

OA affects the hands, lower back, neck, and weight-bearing joints such as the knees, hips, and foot joints. The yearly incidence of OA of the hand is about 50 new cases per 1,000 for persons under age 40, rising to 65 per 1,000 for ages 40-59 and 110 per 1,000 for ages 60 and greater.

OA has been characterized as a slowly evolving degenerative disease with a multifactorial etiology that may differ depending on the joint site. OA occurs when cartilage, the tissue that cushions the ends of the bones within the joints, begins to break down and wear away. In some cases, all of the cartilage may wear away, leaving bones that rub against each other. Arthroscopic studies of early disease have shown synovitis in approximately half of those joints with cartilage damage, suggesting a localized inflammatory reaction in patients with early OA. Furthermore, numerous studies have identified an association between C-reactive protein (CRP) and OA. CRP is an acute phase response protein whose production is stimulated by cytokines, particularly interleukin-6 (IL-6). The relationship between inflammatory processes and elevation in plasma CRP and pro-inflammatory cytokines is well known. CRP has also been related to the inflammatory activity of rheumatoid arthritis.

Symptoms of OA range from stiffness and intermittent mild pain to severe joint pain and impaired biomechanical function. Symptoms can also include fatigue. Although there is no cure for most forms of OA, various therapies can help patients manage symptoms and improve their overall quality of life. Symptomatic treatment of OA traditionally involves administration of non-steroidal anti-inflammatory drugs (NSAIDs), local analgesic therapies, intra-articular corticosteroid injection, and surgery.

Treatment of OA with NSAIDs such as indomethacin, ketoprofen, ibuprofen, acetylsalicylic acid (ASA), and flurbiprofen can relieve pain by reducing local inflammation and attenuating levels of proinflammatory agents. However, long-term NSAID use is compromised by significant gastrointestinal (GI) toxicity. A large multi-center, prospective, observational study involving 1,921 patients with rheumatoid arthritis taking NSAIDs reported that 81% of patients who were hospitalized with serious GI complications had no prior GI problems. This makes it difficult for clinicians to identify patients at risk for GI side-effects. In the United States, it has been conservatively estimated that there are 107,000 annual hospitalizations for NSAID-related GI complications and 16,500 annual NSAID-related deaths among patients with RA or OA. This mortality figure is almost as high as the number of deaths due to asthma, cervical cancer and malignant melanoma combined.

Immunoinflammatory disorders affect millions of individuals and include conditions such as asthma, allergic intraocular inflammatory diseases, arthritis, atopic dermatitis, atopic eczema, diabetes, hemolytic anaemia, inflammatory dermatoses, inflammatory bowel or gastrointestinal disorders (e.g., Crohn's disease and ulcerative colitis), multiple sclerosis, myasthenia gravis, pruritis/inflammation, psoriasis, rheumatoid arthritis, cirrhosis, and systemic lupus erythematosus.

Periodontal disease involves the inflammation, destruction and degeneration of periodontal tissues that surround and support mammalian teeth. These periodontal tissues include the crevicular epithelium, junctional epithelium, external marginal epithelium, gingiva, alveolar bone, periodontal ligament, and cementum. The loss of supporting bone in periodontitis is the latest stage of this progressive disorder and is the major cause of tooth loss in adults.

Periodontal disease is typically classified as gingivitis and periodontitis according to the progress of disease. “Gingivitis” refers to a condition in which inflammation is localized within the gingiva and no lesion occurs in the bone and periodontal ligament, and a pocket is relative pocket. “Periodontitis” refers to a condition in which the inflammation of gingiva reaches the periodontal ligament and alveolar bone, the pocket becomes a periodontal pocket, and the attachment level (the position of attachment) is on the root apex side downward from the cementum-enamel junction. The inflammation prolongs and proceeds toward deep parts with a deepening periodontal pocket. The relationship between inflammatory processes and elevation in plasma C-reactive protein (CRP) and proinflammatory cytokines is well known, and this relationship is observed in periodontitis.

Steroids are known powerful anti-inflammatory agents that have been used in treating musculoskeletal disorders and certain immunoinflammatory disorders. However, chronic administration of anti-inflammatory doses of steroids is also limited by well-known toxicities. For example, prolonged use of steroids has been associated with osteoporosis, high blood pressure, neurological complications, suboptimal immune response, and ocular disturbances, limiting their utility in therapeutic situations. There have been no reports on the use of corticosteroids to treat periodontitis, perhaps because chronic administration of antiinflammatory doses of steroids is limited by well known toxicities. A therapeutic regimen that, for example, retained the potent anti-inflammatory effects of steroids, while limiting the associated toxicities, would be of great benefit to patients with musculoskeletal disorders, periodontal disease, or immunoinflammatory disorders.

### SUMMARY OF THE INVENTION

The invention features methods, compositions, and kits for treating musculoskeletal disorders, periodontal disease, or immunoinflammatory disorders by administering corticosteroids in a split dose regimen.

Accordingly, in a first aspect, the invention features a method for treating an immunoinflammatory disorder in a patient in need thereof by administering to the patient a first dose of a first corticosteroid at a time T₁ followed by a second dose of a second corticosteroid at a time T₂, wherein the time T₁ and the time T₂ are separated by 2 to 10 hours and wherein the first corticosteroid and the second corticosteroid are administered in amounts that together are sufficient to treat the patient.

The invention also features a method for treating an immunoinflammatory disorder in a patient in need thereof by administering to the patient a pharmaceutical composition in unit dosage form including a first dose of a first corticosteroid formulated for immediate release and a second dose of a second corticosteroid formulated for 2 to 10 hour delayed release, wherein the first dose and the second dose are administered in amounts that together are sufficient to treat the patient.

In a related aspect, the invention features a method for treating an immunoinflammatory disorder in a patient in need thereof by administering to the patient a pharmaceutical composition in unit dosage form including a corticosteroid formulated for 2 to 10 hour sustained release, wherein the corticosteroid is from 1 to 30 mg of prednisolone, or an equivalent, equipotent amount of another corticosteroid.

The invention further features a method for treating a disease or condition associated with an increased serum CRP level in a patient in need thereof by administering to the patient a first dose of a first corticosteroid at a time T₁ followed by a second dose of a second corticosteroid at a time T₂, wherein the time T₁ and the time T₂ are separated by 2 to 10 hours and wherein the first dose and the second dose are administered in amounts that together are sufficient to treat the patient.

The invention also features a method for treating a disease or condition associated with an increased serum CRP level in a patient in need thereof by administering to the patient a pharmaceutical composition in unit dosage form including a first dose of a first corticosteroid formulated for immediate release and a second dose of a second corticosteroid formulated for 2 to 10 hour delayed release, wherein the first dose and the second dose are administered in amounts that together are sufficient to treat the patient.

In a related aspect, the invention features a method for treating a disease or condition associated with an increased serum CRP level in a patient in need thereof by administering to the patient a pharmaceutical composition in unit dosage form including a corticosteroid formulated for 2 to 10 hour sustained release, wherein the corticosteroid is from 1 to 30 mg of prednisolone, or an equivalent, equipotent amount of another corticosteroid.

The invention further features a method for reducing the serum C-reactive protein (CRP) level in a patient in need thereof by administering to the patient a first dose of a first corticosteroid at a time T₁ followed by a second dose of a second corticosteroid at a time T₂, wherein the time T₁ and the time T₂ are separated by 2 to 10 hours and wherein the first dose and the second dose are administered in amounts that together are sufficient to reduce the serum CRP level in the patient.

The invention also features a method for reducing the serum C-reactive protein (CRP) level in a patient in need thereof by administering to the patient a pharmaceutical composition in unit dosage form including a first dose of a first corticosteroid formulated for immediate release and a second dose of a second corticosteroid formulated for 2 to 10 hour delayed release, wherein the first dose and the second dose are administered in amounts that together are sufficient to treat the patient.

In a related aspect, the invention features a method for reducing the serum C-reactive protein (CRP) level in a patient in need thereof by administering to the patient a pharmaceutical composition in unit dosage form including a corticosteroid formulated for 2 to 10 hour sustained release, wherein the corticosteroid is from 1 to 30 mg of prednisolone, or an equivalent, equipotent amount of another corticosteroid.

The invention further features a method for treating pain or fatigue associated with a musculoskeletal disorder in a patient in need thereof by administering to the patient a first dose of a first corticosteroid at a time T₁ followed by a second dose of a second corticosteroid at a time T₂, wherein the time T₁ and the time T₂ are separated by 2 to 10 hours and wherein the first dose of a corticosteroid and the second dose of a corticosteroid are administered in amounts that together are sufficient to treat the patient.

The invention also features a method for treating pain or fatigue associated with a musculoskeletal disorder in a patient in need thereof by administering to the patient a pharmaceutical composition in unit dosage form including a first dose of a first corticosteroid formulated for immediate release and a second dose of a second corticosteroid formulated for 2 to 10 hour delayed release, wherein the first dose and the second dose are administered in amounts that together are sufficient to treat the patient.

In a related aspect, the invention features a method for treating pain or fatigue associated with a musculoskeletal disorder in a patient in need thereof by administering to the patient a pharmaceutical composition in unit dosage form including a corticosteroid formulated for 2 to 10 hour sustained release, wherein the corticosteroid is from 1 to 30 mg of prednisolone, or an equivalent, equipotent amount of another corticosteroid.

The invention further features a method for treating tenderness, impairment in mobility, soft tissue swelling, or bony swelling associated with a musculoskeletal disorder in a patient in need thereof by administering to the patient a first dose of a first corticosteroid at a time T₁ followed by a second dose of a second corticosteroid at a time T₂, wherein the time T₁ and the time T₂ are separated by 2 to 10 hours and wherein the first dose of a corticosteroid and the second dose of a corticosteroid are administered in amounts that together are sufficient to treat the patient.

The invention also features a method for treating tenderness, impairment in mobility, soft tissue swelling, or bony swelling associated with a musculoskeletal disorder in a patient in need thereof by administering to the patient a pharmaceutical composition in unit dosage form including a first dose of a first corticosteroid formulated for immediate release and a second dose of a second corticosteroid formulated for 2 to 10 hour delayed release, wherein the first dose and the second dose are administered in amounts that together are sufficient to treat the patient.

In a related aspect, the invention features a method for treating tenderness, impairment in mobility, soft tissue swelling, or bony swelling associated with a musculoskeletal disorder in a patient in need thereof by administering to the patient a pharmaceutical composition in unit dosage form including a corticosteroid formulated for 2 to 10 hour sustained release, wherein the corticosteroid is from 1 to 30 mg of prednisolone, or an equivalent, equipotent amount of another corticosteroid.

The invention further features a method for treating a Group A musculoskeletal disorder in a patient in need thereof by administering to the patient a first dose of a first corticosteroid at a time T₁ followed by a second dose of a second corticosteroid at a time T₂, wherein the time T₁ and the time T₂ are separated by 2 to 10 hours and wherein the first dose of a corticosteroid and the second dose of a corticosteroid are administered in amounts that together are sufficient to treat the patient.

The invention also features a method for treating a Group A musculoskeletal disorder in a patient in need thereof by administering to the patient a pharmaceutical composition in unit dosage form including a first dose of a first corticosteroid formulated for immediate release and a second dose of a second corticosteroid formulated for 2 to 10 hour delayed release, wherein the first dose and the second dose are administered in amounts that together are sufficient to treat the patient.

In a related aspect, the invention features a method for treating a Group A musculoskeletal disorder in a patient in need thereof by administering to the patient a pharmaceutical composition in unit dosage form including a corticosteroid formulated for 2 to 10 hour sustained release, wherein the corticosteroid is from 1 to 30 mg of prednisolone, or an equivalent, equipotent amount of another corticosteroid.

The invention further features a method for treating a Group B musculoskeletal disorder in a patient in need thereof by administering to the patient a first dose of a first corticosteroid at a time T₁ followed by a second dose of a second corticosteroid at a time T₂, wherein the time T₁ and the time T₂ are separated by 2 to 10 hours and wherein the first dose of a corticosteroid and the second dose of a corticosteroid are administered in amounts that together are sufficient to treat the patient.

The invention also features a method for treating a Group B musculoskeletal disorder in a patient in need thereof by administering to the patient a pharmaceutical composition in unit dosage form including a first dose of a first corticosteroid formulated for immediate release and a second dose of a second corticosteroid formulated for 2 to 10 hour delayed release, wherein the first dose and the second dose are administered in amounts that together are sufficient to treat the patient.

In a related aspect, the invention features a method for treating a Group B musculoskeletal disorder in a patient in need thereof by administering to the patient a pharmaceutical composition in unit dosage form including a corticosteroid formulated for 2 to 10 hour sustained release, wherein the corticosteroid is from 1 to 30 mg of prednisolone, or an equivalent, equipotent amount of another corticosteroid.

The invention further features a method for treating pain in a patient in need thereof by administering to the patient a first dose of a first corticosteroid at a time T₁ followed by a second dose of a second corticosteroid at a time T₂, wherein the time T₁ and the time T₂ are separated by 2 to 10 hours and wherein the first corticosteroid and the second corticosteroid are administered in amounts that together are sufficient to treat the patient.

The invention also features a method for treating pain in a patient in need thereof by administering to the patient a pharmaceutical composition in unit dosage form including a first dose of a first corticosteroid formulated for immediate release and a second dose of a second corticosteroid formulated for 2 to 10 hour delayed release, wherein the first dose and the second dose are administered in amounts that together are sufficient to treat the patient.

In a related aspect, the invention features a method for treating pain in a patient in need thereof by administering to the patient a pharmaceutical composition in unit dosage form including a corticosteroid formulated for 2 to 10 hour sustained release, wherein the corticosteroid is from 1 to 30 mg of prednisolone, or an equivalent, equipotent amount of another corticosteroid.

The invention further features a method for treating periodontal disease in a patient in need thereof by administering to the patient a first dose of a first corticosteroid at a time T₁ followed by a second dose of a second corticosteroid at a time T₂, wherein the time T₁ and the time T₂ are separated by 2 to 10 hours and wherein the first dose and the second dose are administered in amounts that together are sufficient to treat the patient.

The invention also features a method for treating periodontal disease in a patient in need thereof by administering to the patient a pharmaceutical composition in unit dosage form including a first dose of a first corticosteroid formulated for immediate release and a second dose of a second corticosteroid formulated for 2 to 10 hour delayed release, wherein the first dose and the second dose are administered in amounts that together are sufficient to treat the patient.

In a related aspect, the invention features a method for treating periodontal disease in a patient in need thereof by administering to the patient a pharmaceutical composition in unit dosage form including a corticosteroid formulated for 2 to 10 hour sustained release, wherein the corticosteroid is from 1 to 30 mg of prednisolone, or an equivalent, equipotent amount of another corticosteroid.

The invention further features a kit including (i) a first composition including a first dose of a first corticosteroid; (ii) a second composition including a second dose of a second corticosteroid; and (iii) instructions for administering the first composition at a time T₁ followed by the second composition at a time T₂ to a patient diagnosed with or at risk of developing an immunoinflammatory disorder, wherein the time T₁ and the time T₂ are separated by 2 to 10 hours.

The invention also features a kit including (i) a pharmaceutical composition in unit dosage form including a first dose of a first corticosteroid formulated for immediate release and a second dose of a second corticosteroid formulated for 2 to 10 hour delayed release; and (ii) instructions for administering the pharmaceutical composition to a patient diagnosed with or at risk of developing an immunoinflammatory disorder.

In a related aspect, the invention features a kit including (i) a pharmaceutical composition in unit dosage form including a corticosteroid formulated for 2 to 10 hour sustained release, wherein the corticosteroid is from 1 to 30 mg of prednisolone, or an equivalent, equipotent amount of another corticosteroid; and (ii) instructions for administering the pharmaceutical composition to a patient diagnosed with or at risk of developing an immunoinflammatory disorder.

The invention further features a kit including (i) a first composition including a first dose of a first corticosteroid; (ii) a second composition including a second dose of a second corticosteroid; and (iii) instructions for administering the first composition at a time T₁ followed by the second composition at a time T₂ to a patient diagnosed with or at risk of developing a disease or condition associated with an increased serum CRP levels, wherein the time T₁ and the time T₂ are separated by 2 to 10 hours.

The invention also features a kit including (i) a pharmaceutical composition in unit dosage form including a first dose of a first corticosteroid formulated for immediate release and a second dose of a second corticosteroid formulated for 2 to 10 hour delayed release; and (ii) instructions for administering the pharmaceutical composition to a patient diagnosed with or at risk of developing a disease or condition associated with an increased serum CRP levels.

In a related aspect, the invention features a kit including (i) a pharmaceutical composition in unit dosage form including a corticosteroid formulated for 2 to 10 hour sustained release, wherein the corticosteroid is from 1 to 30 mg of prednisolone, or an equivalent, equipotent amount of another corticosteroid; and (ii) instructions for administering the pharmaceutical composition to a patient diagnosed with or at risk of developing a disease or condition associated with an increased serum CRP levels.

The invention further features a kit including (i) a first composition including a first dose of a first corticosteroid; (ii) a second composition including a second dose of a second corticosteroid; and (iii) instructions for administering the first composition at a time T₁ followed by the second composition at a time T2 to a patient for the treatment of pain or fatigue associated with a musculoskeletal disorder, wherein the time T₁ and the time T₂ are separated by 2 to 10 hours.

The invention also features a kit including (i) a pharmaceutical composition in unit dosage form including a first dose of a first corticosteroid formulated for immediate release and a second dose of a second corticosteroid formulated for 2 to 10 hour delayed release; and (ii) instructions for administering the pharmaceutical composition to a patient for the treatment of pain or fatigue associated with a musculoskeletal disorder.

In a related aspect, the invention features a kit including (i) a pharmaceutical composition in unit dosage form including a corticosteroid formulated for 2 to 10 hour sustained release, wherein the corticosteroid is from 1 to 30 mg of prednisolone, or an equivalent, equipotent amount of another corticosteroid; and (ii) instructions for administering the pharmaceutical composition to a patient for the treatment of pain or fatigue associated with a musculoskeletal disorder.

The invention further features a kit including (i) a first composition including a first dose of a first corticosteroid; (ii) a second composition including a second dose of a second corticosteroid; and (iii) instructions for administering the first composition at a time T₁ followed by the second composition at a time T₂ to a patient for the treatment of tenderness, impairment in mobility, soft tissue swelling, or bony swelling associated with a musculoskeletal disorder, wherein the time T₁ and the time T₂ are separated by 2 to 10 hours.

The invention also features a kit including (i) a pharmaceutical composition in unit dosage form including a first dose of a first corticosteroid formulated for immediate release and a second dose of a second corticosteroid formulated for 2 to 10 hour delayed release; and (ii) instructions for administering the pharmaceutical composition to a patient for the treatment of tenderness, impairment in mobility, soft tissue swelling, or bony swelling associated with a musculoskeletal disorder.

In a related aspect, the invention features a kit including (i) a pharmaceutical composition in unit dosage form including a corticosteroid formulated for 2 to 10 hour sustained release, wherein the corticosteroid is from 1 to 30 mg of prednisolone, or an equivalent, equipotent amount of another corticosteroid; and (ii) instructions for administering the pharmaceutical composition to a patient for the treatment of tenderness, impairment in mobility, soft tissue swelling, or bony swelling associated with a musculoskeletal disorder.

The invention further features a kit including (i) a first composition including a first dose of a first corticosteroid; (ii) a second composition including a second dose of a second corticosteroid; and (iii) instructions for administering the first composition at a time T₁ followed by the second composition at a time T₂ to a patient diagnosed with or at risk of developing a Group A musculoskeletal disorder, wherein the time T₁ and the time T₂ are separated by 2 to 10 hours.

The invention also features a kit including (i) a pharmaceutical composition in unit dosage form including a first dose of a first corticosteroid formulated for immediate release and a second dose of a second corticosteroid formulated for 2 to 10 hour delayed release; and (ii) instructions for administering the pharmaceutical composition to a patient diagnosed with or at risk of developing a Group A musculoskeletal disorder.

In a related aspect, the invention features a kit including (i) a pharmaceutical composition in unit dosage form including a corticosteroid formulated for 2 to 10 hour sustained release, wherein the corticosteroid is from 1 to 30 mg of prednisolone, or an equivalent, equipotent amount of another corticosteroid; and (ii) instructions for administering the pharmaceutical composition to a patient diagnosed with or at risk of developing a Group A musculoskeletal disorder.

The invention further features a kit including (i) a first composition including a first dose of a first corticosteroid; (ii) a second composition including a second dose of a second corticosteroid; and (iii) instructions for administering the first composition at a time T₁ followed by the second composition at a time T₂ to a patient diagnosed with or at risk of developing a Group B musculoskeletal disorder, wherein the time T₁ and the time T₂ are separated by 2 to 10 hours.

The invention also features a kit including (i) a pharmaceutical composition in unit dosage form including a first dose of a first corticosteroid formulated for immediate release and a second dose of a second corticosteroid formulated for 2 to 10 hour delayed release; and (ii) instructions for administering the pharmaceutical composition to a patient diagnosed with or at risk of developing a Group B musculoskeletal disorder.

In a related aspect, the invention features a kit including (i) a pharmaceutical composition in unit dosage form including a corticosteroid formulated for 2 to 10 hour sustained release, wherein the corticosteroid is from 1 to 30 mg of prednisolone, or an equivalent, equipotent amount of another corticosteroid; and (ii) instructions for administering the pharmaceutical composition to a patient diagnosed with or at risk of developing a Group B musculoskeletal disorder.

The invention further features a kit including (i) a first composition including a first dose of a first corticosteroid; (ii) a second composition including a second dose of a second corticosteroid; and (iii) instructions for administering the first composition at a time T₁ followed by the second composition at a time T₂ to a patient for the treatment of pain, wherein the time T₁ and the time T₂ are separated by 2 to 10 hours.

The invention also features a kit including (i) a pharmaceutical composition in unit dosage form including a first dose of a first corticosteroid formulated for immediate release and a second dose of a second corticosteroid formulated for 2 to 10 hour delayed release; and (ii) instructions for administering the pharmaceutical composition to a patient for the treatment of pain.

In a related aspect, the invention features a kit including (i) a pharmaceutical composition in unit dosage form including a corticosteroid formulated for 2 to 10 hour sustained release, wherein the corticosteroid is from 1 to 30 mg of prednisolone, or an equivalent, equipotent amount of another corticosteroid; and (ii) instructions for administering the pharmaceutical composition to a patient for the treatment of pain.

The invention further features a kit including (i) a first composition including a first dose of a first corticosteroid; (ii) a second composition including a second dose of a second corticosteroid; and (iii) instructions for administering the first composition at a time T₁ followed by the second composition at a time T₂ to a patient for the treatment of periodontal disease, wherein the time T₁ and the time T₂ are separated by 2 to 10 hours.

The invention also features a kit including (i) a pharmaceutical composition in unit dosage form including a first dose of a first corticosteroid formulated for immediate release and a second dose of a second corticosteroid formulated for 2 to 10 hour delayed release; and (ii) instructions for administering the pharmaceutical composition to a patient for the treatment of periodontal disease.

The invention further features a pharmaceutical composition formulated for oral administration in unit dosage form including a first dose of a first corticosteroid formulated for immediate release and a second dose of a second corticosteroid formulated for 2 to 10 hour delayed release. In one embodiment, the first corticosteroid and the second corticosteroid are the only active pharmaceutical ingredients present in the pharmaceutical composition. In another embodiment, the first corticosteroid and the second corticosteroid are the same.

In one embodiment of the methods, kits, and compositions of the invention, the first dose and the second dose together are equal to or less than 30 mg of prednisolone or an equivalent, equipotent amount of another corticosteroid. Desirably, the first dose and the second dose together are equal to or less than 28 mg, 26 mg, 24 mg, 22 mg, 20 mg, 18 mg, 16 mg, 14 mg, 12 mg, 10 mg, 9 mg, 8.5 mg, 8 mg, 7.5 mg, 7 mg, 6.5 mg, 6 mg, 5.5 mg, 5 mg, 4.5 mg, 4 mg, 3.5 mg, 3 mg, 2.9 mg, 2.8 mg, 2.7 mg, 2.6 mg, 2.5 mg, 2.4 mg, 2.3 mg, 2.2 mg, 2.1 mg, or 2.0 mg.

In another embodiment of the methods, kits, and compositions of the invention, the ratio (w/w) of corticosteroid in the first dose to the second dose is from 1:10 to 10:1, 1:5 to 5:1, 1:4 to 4:1, or 1:3 to 3:1. Desirably, the ratio is 1:2 to 2:1, 1.2:2, 1.4:2, 1.6:2, 1.8:2, 1:1, 2:1.8, 2:1.6, 2:1.4, or 2:1.2.

In one embodiment of the methods, kits, and compositions of the invention, the first dose is from 0.25 mg to 10 mg of prednisolone or an equivalent, equipotent amount of another corticosteroid and the second dose is from 5 mg to 25 mg of prednisolone or an equivalent, equipotent amount of another corticosteroid. In certain embodiments, the first dose is from 0.5 mg to 9 mg, 0.25 mg to 3 mg, 2.5 mg to 10 mg, 0.5 mg to 4 mg, 0.5 mg to 1.5 mg, 0.75 mg to 1.25 mg, 1.0 mg to 10 mg, 1.0 mg to 9.0 mg, 1.0 mg to 8.0 mg, 1.0 mg to 7.0 mg, 1.0 mg to 6.0 mg, 1.0 mg to 5.0 mg, 1.0 mg to 4.0 mg, 1.0 mg to 3.0 mg, 2.0 mg to 8 mg, 2.0 mg to 6 mg, 3.0 mg to 10 mg, 0.75 mg to 2.5 mg, or 0.25 mg to 1.75 mg of prednisolone or an equivalent, equipotent amount of another corticosteroid. In certain embodiments, the second dose is from 5 mg to 24 mg, 5 mg to 22 mg, 5 mg to 20 mg, 5 mg to 18 mg, 5 mg to 16 mg, 5 mg to 14 mg, 5 mg to 12 mg, 5 mg to 10 mg, 5 mg to 8 mg, 6 mg to 14 mg, 8 mg to 16 mg, 10 mg to 18 mg, 12 mg to 20 mg, or 14 mg to 22 mg of prednisolone or an equivalent, equipotent amount of another corticosteroid. Desirably, the first dose is 2.5 mg of prednisolone or an equivalent, equipotent amount of another corticosteroid and the second dose is 5.0 mg of prednisolone or an equivalent, equipotent amount of another corticosteroid; or the first dose is 5 mg of prednisolone or an equivalent, equipotent amount of another corticosteroid and the second dose is 2.5 mg of prednisolone or an equivalent, equipotent amount of another corticosteroid.

In one embodiment of the methods, kits, and compositions of the invention, the first dose is from 0.25 mg to 5 mg of prednisolone or an equivalent, equipotent amount of another corticosteroid and the second dose is from 0.25 mg to 5 mg of prednisolone or an equivalent, equipotent amount of another corticosteroid. In certain embodiments, the first dose is from 0.5 mg to 5 mg, 0.25 mg to 3 mg, 2.5 mg to 5 mg, 0.5 mg to 4 mg, 0.5 mg to 1.5 mg, 0.75 mg to 1.25 mg, 1.0 mg to 5.0 mg, 1.0 mg to 4.0 mg, 1.0 mg to 3.0 mg, 1.0 mg to 2.0 mg, 2.0 mg to 4 mg, 2.0 mg to 3 mg, 3.0 mg to 5 mg, 0.75 mg to 2.5 mg, or even 0.25 mg to 1.75 mg of prednisolone or an equivalent, equipotent amount of another corticosteroid. In certain embodiments, the second dose is from 0.5 mg to 5 mg, 0.25 mg to 3 mg, 2.5 mg to 5 mg, 0.5 mg to 4 mg, 0.5 mg to 1.5 mg, 0.75 mg to 1.25 mg, 1.0 mg to 5.0 mg, 1.0 mg to 4.0 mg, 1.0 mg to 3.0 mg, 1.0 mg to 2.0 mg, 2.0 mg to 4 mg, 2.0 mg to 3 mg, 3.0 mg to 5 mg, 0.75 mg to 2.5 mg, or even 0.25 mg to 1.75 mg of prednisolone or an equivalent, equipotent amount of another corticosteroid. Desirably, the first dose is 1.0 mg of prednisolone or an equivalent, equipotent amount of another corticosteroid and the second dose is 2.0 mg of prednisolone or an equivalent, equipotent amount of another corticosteroid; or the first dose is 2.0 mg of prednisolone or an equivalent, equipotent amount of another corticosteroid and the second dose is 1.0 mg of prednisolone or an equivalent, equipotent amount of another corticosteroid.

In one embodiment of the methods, kits, and compositions of the invention including a second dose of corticosteroid formulated for delayed release, the second dose can be formulated for 2 to 9 hour, 2 to 8 hour, 2 to 7 hour, 2 to 6 hour, 3 to 8 hour, 3 to 7 hour, 3 to 6 hour, 4 to 9 hour, 4 to 8 hour, 4 to 7 hour, 4 to 6 hour, 2 to 5 hour, or 2 to 4 hour delayed release.

In one embodiment of the methods, kits, and compositions of the invention including for administering to the patient a first dose of a first corticosteroid at a time T₁ followed by a second dose of a second corticosteroid at a time T₂, the time T₁ and the time T₂ can be separated by 2 to 9 hours, 2 to 8 hours, 2 to 7 hours, 2 to 6 hours, 2 to 5 hours, 2 to 4 hours, 3 to 8 hours, 3 to 7 hours, 3 to 6 hours, 3 to 5 hours, 4 to 10 hours, 4 to 8 hours, 4 to 6 hours, 5 to 10 hours, 5 to 9 hours, 5 to 8 hours, 5 to 7 hours, or 6 to 10 hours. In certain embodiments, time T₁ is in the morning and time T₂ is after noon. In other embodiments, time T₂ is in the morning and time T₁ is after noon.

In yet another embodiment of the methods, kits, and compositions of the invention including for administering to the patient a first dose of a first corticosteroid at a time T₁ followed by a second dose of a second corticosteroid at a time T₂, the first dose is formulated for immediate release and the second dose is formulated for delayed release, wherein the first dose and the second dose are each formulated in unit dosage form for oral administration.

In a related aspect, the invention features a pharmaceutical composition formulated for oral administration in unit dosage form including a corticosteroid formulated for 2 to 10 hour sustained release, wherein the corticosteroid is from 1 to 30 mg of prednisolone, or an equivalent, equipotent amount of another corticosteroid.

In certain embodiments of the methods, kits, and compositions of the invention directed to sustained release corticosteroid formulations, the corticosteroid is from 1 to 20 mg, 1 to 15 mg, 1 to 10 mg, 3 to 10 mg, 5 to 10 mg, 1 to 5 mg, 1 to 4.5 mg, 1 to 4 mg, 1 to 3.5 mg, 1 to 3 mg, 1 to 2.5 mg, or 10 mg, 9 mg, 8 mg, 7 mg, 6 mg, 5 mg, 4.5 mg, 4 mg, 3.5 mg, 3 mg, 2.5 mg, 2 mg, or 1.5 mg of prednisolone, or an equivalent, equipotent amount of another corticosteroid. In other embodiments, the corticosteroid is formulated for 2 to 12 hour, 2 to 9 hour, 2 to 8 hour, 2 to 7 hour, 2 to 6 hour, 3 to 9 hour, 3 to 8 hour, 3 to 7 hour, 3 to 6 hour, 4 to 10 hour, 4 to 9 hour, 4 to 8 hour, or 4 to 7 hour sustained release. In still another embodiment, the sustained release corticosteroid is the only active pharmaceutical ingredient being administered to the patient.

In still another embodiment of the methods, kits, and compositions of the invention, the kit, composition or method includes administering a DMARD to the patient, or any other therapeutic agent described herein. Additionally, combination therapeutics containing a corticosteroid and one or more active ingredient are known in the art (see, e.g., U.S. Patent Application Publication Nos. 2004-0229849; 2005-0153947; 2005-0187200; 2005-0187203; 2005-0192261; 2006-0100181; 2006-0148770; 2006-0234911; 2006-0286177; and 2007-0010502, each of which is hereby incorporated by reference). The corticosteroid-containing compositions described herein can also contain these additional active ingredients and can be used to treat the diseases and conditions recited herein.

In one particular embodiment of the methods, kits, and compositions of the invention, the first corticosteroid and the second corticosteroid are the same.

In one particular embodiment of the methods, kits, and compositions of the invention, the first corticosteroid and the second corticosteroid are the only active pharmaceutical ingredients being administered to the patient.

In any of the above aspects, the first corticosteroid and the second corticosteroid, and sustained release corticosteroid can be selected from, without limitation, algestone, 6-alpha-fluoroprednisolone, 6-alpha-methylprednisolone, 6-alpha-methylprednisolone 21-acetate, 6-alpha-methylprednisolone 21-hemisuccinate sodium salt, 6-alpha-9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-17-valerate, budesonide, clobetasol, clobetasol propionate, clobetasone, clocortolone, clocortolone pivalate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, flupredidene, fluprednisolone, flurandrenolide, formocortal, halcinonide, halometasone, halopredone, hyrcanoside, hydrocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methylprednisolone, methylprednisolone acetate, methylprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-21-acetate, prednisolone-21(beta-D-glucuronide), prednisone, prednylidene, procinonide, tralonide, triamcinolone, triamcinolone acetonide, triamcinolone acetonide 21-palmitate, triamcinolone diacetate, triamcinolone hexacetonide, and wortmannin. In one embodiment, both of the first corticosteroid and the second corticosteroid are prednisolone.

The methods, kits, and compositions of the invention can be used to treat an immunoinflammatory disorder, including, without limitation, rheumatoid arthritis, Crohn's disease, ulcerative colitis, asthma, chronic obstructive pulmonary disease, polymylagia rheumatica, giant cell arteritis, systemic lupus erythematosus, atopic dermatitis, multiple sclerosis, myasthenia gravis, psoriasis, ankylosing spondylitis, cirrhosis, and psoriatic arthritis.

The methods, kits, and compositions of the invention can be used to treat a disease or condition associated with an increased serum CRP level, including, without limitation, cardiovascular disease, hypertension, colon cancer, lymphoma, and sarcoma.

The methods, kits, and compositions of the invention can be used to treat a Group A musculoskeletal disorder, including, without limitation, arthritis, ankylosing spondylitis, Behcet's disease, bursitis, dermatomyositis, fasciitis, fibromyalgia, lupus, myositis, myositis ossificans, necrotizing fasciitis, polymyalgia rheumatica, psoriatic arthritis, relapsing polychondritis, rheumatic fever, scleroderma, Sjögren's syndrome, Still's disease, and Wegener's granulomatosis.

The methods, kits, and compositions of the invention can be used to treat a Group B musculoskeletal disorder, including, without limitation, acquired hyperostosis syndrome, acromegaly, chronic fatigue syndrome, congenital hypothyroidism, dentigerous cyst, diffuse idiopathic skeletal hyperostosis, Dupuytren's contracture, eosinophilia-myalgia syndrome, Felty's syndrome, hallux valgus, Kabuki make-up syndrome, Legg-Perthes disease, Lyme disease, Melas syndrome, neurogenic arthropathy, osteitis deformans, osteochondritis, osteomalacia, osteomyelitis, osteonecrosis, osteoporosis, Paget's disease, Pierre Robin syndrome, polymyositis, postpoliomyelitis syndrome, pseudogout, Reiter disease, renal osteodystrophy, rhabdomyolysis, Sever's disease (calceneal apophysitis), spinal stenosis, synovitis, tendinopathy, tennis elbow, tenosynovitis, and Tietze's syndrome.

The methods, kits, and compositions of the invention can be used to treat pain, including, without limitation, inflammatory pain (e.g., postoperative pain, post-traumatic pain, arthritic pain, or pain associated with damage to joints, muscle, and tendons), neuropathic pain (e.g., trauma, surgery, herniation of an intervertebral disk, spinal cord injury, shingles, HIV/AIDS, late-stage cancer, amputation, and carpal tunnel syndrome), or nociceptive pain (e.g., nociceptive pain caused by surgery, labor, sprains, bone fractures, burns, bumps, bruises, injections, dental procedures, biopsies, or obstructions). In certain embodiments, the pain being treated is dysfunctional pain caused by fibromyalgia, tension type headache, irritable bowel disorders, or migraine.

The methods, kits, and compositions of the invention can be used to treat periodontal disease, including, without limitation, periodontitis and gingivitis.

As used herein, “split dose” refers to (1) dosing regimens in which one or more corticosteroids are administered to a patient at least twice daily; (2) once daily administration of a pharmaceutical composition containing one or more corticosteroids in which a portion of the corticosteroid is formulated for immediate release and a portion of the corticosteroid is formulated for delayed release; and (3) once daily administration of a pharmaceutical composition containing a corticosteroid formulated for sustained release. With respect to the second embodiment, although the corticosteroid is administered once daily, the corticosteroid is delivered to the patient in a split dose fashion as a result of using two different formulations in a single pharmaceutical composition. With respect to the third embodiment, although the corticosteroid is administered once daily, the corticosteroid is delivered to the patient in a manner that provides sustained low dose exposure that could only otherwise be achieved by administering multiple dosings in small amounts throughout the course of a day.

By “corticosteroid” is meant any naturally occurring or synthetic compound characterized by a hydrogenated cyclopentanoperhydrophenanthrene ring system. Naturally occurring corticosteroids are generally produced by the adrenal cortex. Synthetic corticosteroids may be halogenated. Exemplary corticosteroids are described herein.

By an “equivalent, equipotent amount” is meant a dosage of a corticosteroid that produces the same anti-inflammatory effect in a patient as a recited dosage of prednisolone.

Corticosteroids useful in the methods, compositions, and kits of the invention include, e.g., algestone, 6-alpha-fluoroprednisolone, 6-alpha-methylprednisolone, 6-alpha-methylprednisolone 21-acetate, 6-alpha-methylprednisolone 21-hemisuccinate sodium salt, 6-alpha-9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-17-valerate, budesonide, clobetasol, clobetasol propionate, clobetasone, clocortolone, clocortolone pivalate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, flupredidene, fluprednisolone, flurandrenolide, formocortal, halcinonide, halometasone, halopredone, hyrcanoside, hydrocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methylprednisolone, methylprednisolone acetate, methylprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-21-acetate, prednisolone-21(beta-D-glucuronide), prednisone, prednylidene, procinonide, tralonide, triamcinolone, triamcinolone acetonide, triamcinolone acetonide 21-palmitate, triamcinolone diacetate, triamcinolone hexacetonide, and wortmannin. Particularly desirable corticosteroids are prednisolone, cortisone, dexamethasone, hydrocortisone, methylprednisolone, fluticasone, prednisone, triamcinolone, and diflorasone.

As used herein, the term “treating” refers to administering a pharmaceutical composition for prophylactic and/or therapeutic purposes. To “prevent disease” refers to prophylactic treatment of a subject who is not yet ill, but who is susceptible to, or otherwise at risk of, a particular disease. To “treat disease” or use for “therapeutic treatment” refers to administering treatment to a subject already suffering from a disease to improve or stabilize the subject's condition. Thus, in the claims and embodiments, treating is the administration to a subject either for therapeutic or prophylactic purposes.

By “an amount sufficient” is meant the amount of a compound, in a combination of the invention, required to treat or prevent a disease or condition in a clinically relevant manner. A sufficient amount of an active compound used to practice the present invention for therapeutic treatment of particular diseases and conditions caused varies depending upon the manner of administration, the age, body weight, and general health of the patient. Ultimately, the prescribers will decide the appropriate amount and dosage regimen.

The term “periodontal disease” encompasses a variety of conditions, including gingivitis and periodontitis, as well as diseases of tissues that surround and support teeth, including the gingiva, cementum, periodontal ligament, alveolar process bone, and dental supporting bone.

By “musculoskeletal disorder” is meant an immune system-related disorder of the muscles, ligaments, bones, joints, cartilage, or other connective tissue. Among the most commonly-occurring musculoskeletal disorders are various forms of arthritis, e.g., osteoarthritis, rheumatoid arthritis, juvenile rheumatoid arthritis, and gout. Other musculoskeletal disorders include acquired hyperostosis syndrome, acromegaly, ankylosing spondylitis, Behcet's disease, bone diseases, bursitis, cartilage diseases, chronic fatigue syndrome, compartment syndromes, congenital hypothyroidism, congenital myopathies, dentigerous cyst, dermatomyositis, diffuse idiopathic skeletal hyperostosis, Dupuytren's contracture, eosinophilia-myalgia syndrome, fasciitis, Felty's syndrome, fibromyalgia, hallux valgus, infectious arthritis, joint diseases, Kabuki make-up syndrome, Legg-Perthes disease, lupus, Lyme disease, Melas syndrome, metabolic bone diseases, mitochondrial myopathies, mixed connective tissue disease, muscular diseases, muscular dystrophies, musculoskeletal abnormalities, musculoskeletal diseases, myositis, myositis ossificans, necrotizing fasciitis, neurogenic arthropathy, osteitis deformans, osteochondritis, osteomalacia, osteomyelitis, osteonecrosis, osteoporosis, Paget's disease, Pierre Robin syndrome, polymyalgia rheumatica, polymyositis, postpoliomyelitis syndrome, pseudogout, psoriatic arthritis, reactive arthritis, Reiter disease, relapsing polychondritis, renal osteodystrophy, rhabdomyolysis, rheumatic diseases, rheumatic fever, scleroderma, Sever's disease (calceneal apophysitis), Sjögren's syndrome, spinal diseases, spinal stenosis, Still's disease, synovitis, temporomandibular joint disorders, tendinopathy, tennis elbow, tenosynovitis, Tietze's syndrome, and Wegener's granulomatosis.

By “Group A musculoskeletal disorder” is meant arthritis (e.g., osteoarthritis, rheumatoid arthritis, juvenile rheumatoid arthritis, or gout), ankylosing spondylitis, Behcet's disease, bursitis, dermatomyositis, fasciitis, fibromyalgia, lupus, myositis, myositis ossificans, necrotizing fasciitis, polymyalgia rheumatica, psoriatic arthritis, relapsing polychondritis, rheumatic fever, scleroderma, Sjögren's syndrome, Still's disease, or Wegener's granulomatosis.

By “Group B musculoskeletal disorder” is meant an immune system-related disorder of the muscles, ligaments, bones, joints, cartilage, or other connective tissue that is not a Group A musculoskeletal disorder. Exemplary Group B musculoskeletal disorders are acquired hyperostosis syndrome, acromegaly, chronic fatigue syndrome, congenital hypothyroidism, dentigerous cyst, diffuse idiopathic skeletal hyperostosis, Dupuytren's contracture, eosinophilia-myalgia syndrome, Felty's syndrome, hallux valgus, Kabuki make-up syndrome, Legg-Perthes disease, Lyme disease, Melas syndrome, neurogenic arthropathy, osteitis deformans, osteochondritis, osteomalacia, osteomyelitis, osteonecrosis, osteoporosis, Paget's disease, Pierre Robin syndrome, polymyositis, postpoliomyelitis syndrome, pseudogout, Reiter disease, renal osteodystrophy, rhabdomyolysis, Sever's disease (calceneal apophysitis), spinal stenosis, synovitis, tendinopathy, tennis elbow, tenosynovitis, and Tietze's syndrome.

By “immunoinflammatory disorder” is meant to encompass a variety of conditions, including autoimmune diseases, proliferative skin diseases, and inflammatory dermatoses. Immunoinflammatory disorders result in the destruction of healthy tissue by an inflammatory process, deregulation of the immune system, and unwanted proliferation of cells. Examples of immunoinflammatory disorders are acne vulgaris; acute respiratory distress syndrome; Addison's disease; allergic rhinitis; allergic intraocular inflammatory diseases, ANCA-associated small-vessel vasculitis; ankylosing spondylitis; arthritis, asthma; atherosclerosis; atopic dermatitis; autoimmune hemolytic anemia; autoimmune hepatitis; Behcet's disease; Bell's palsy; bullous pemphigoid; cerebral ischaemia; chronic obstructive pulmonary disease; cirrhosis; Cogan's syndrome; contact dermatitis; COPD; Crohn's disease; Cushing's syndrome; dermatomyositis; diabetes mellitus; discoid lupus erythematosus; eosinophilic fasciitis; erythema nodosum; exfoliative dermatitis; fibromyalgia; focal glomerulosclerosis; giant cell arteritis; gout; gouty arthritis; graft-versus-host disease; hand eczema; Henoch-Schonlein purpura; herpes gestationis; hirsutism; idiopathic cerato-scleritis; idiopathic pulmonary fibrosis; idiopathic thrombocytopenic purpura; inflammatory bowel or gastrointestinal disorders, inflammatory dermatoses; lichen planus; lupus nephritis; lymphomatous tracheobronchitis; macular edema; multiple sclerosis; myasthenia gravis; myositis; osteoarthritis; pancreatitis; pemphigoid gestationis; pemphigus vulgaris; polyarteritis nodosa; polymyalgia rheumatica; pruritus scroti; pruritis/inflammation, psoriasis; psoriatic arthritis; rheumatoid arthritis; relapsing polychondritis; rosacea caused by sarcoidosis; rosacea caused by scleroderma; rosacea caused by Sweet's syndrome; rosacea caused by systemic lupus erythematosus; rosacea caused by urticaria; rosacea caused by zoster-associated pain; sarcoidosis; scleroderma; segmental glomerulosclerosis; septic shock syndrome; shoulder tendinitis or bursitis; Sjogren's syndrome; Still's disease; stroke-induced brain cell death; Sweet's disease; systemic lupus erythematosus; systemic sclerosis; Takayasu's arteritis; temporal arteritis; toxic epidermal necrolysis; tuberculosis; type-1 diabetes; ulcerative colitis; uveitis; vasculitis; and Wegener's granulomatosis. “Non-dermal inflammatory disorders” include, for example, rheumatoid arthritis, inflammatory bowel disease, asthma, and chronic obstructive pulmonary disease.

“Dermal inflammatory disorders” or “inflammatory dermatoses” include, for example, psoriasis, acute febrile neutrophilic dermatosis, eczema (e.g., asteatotic eczema, dyshidrotic eczema, vesicular palmoplantar eczema), balanitis circumscripta plasmacellularis, balanoposthitis, Behcet's disease, erythema annulare centrifugum, erythema dyschromicum perstans, erythema multiforme, granuloma annulare, lichen nitidus, lichen planus, lichen sclerosus et atrophicus, lichen simplex chronicus, lichen spinulosus, nummular dermatitis, pyoderma gangrenosum, sarcoidosis, subcorneal pustular dermatosis, urticaria, and transient acantholytic dermatosis.

“Non-dermal inflammatory disorders” include, for example, rheumatoid arthritis, inflammatory bowel disease, asthma, and chronic obstructive pulmonary disease.

By “proliferative skin disease” is meant a benign or malignant disease that is characterized by accelerated cell division in the epidermis or dermis. Examples of proliferative skin diseases are psoriasis, atopic dermatitis, non-specific dermatitis, primary irritant contact dermatitis, allergic contact dermatitis, basal and squamous cell carcinomas of the skin, lamellar ichthyosis, epidermolytic hyperkeratosis, premalignant keratosis, acne, and seborrheic dermatitis.

As will be appreciated by one skilled in the art, a particular disease, disorder, or condition may be characterized as being both musculoskeletal and immunoinflammatory. An example of such a disease is osteoarthritis.

The term “pain” is used herein in the broadest sense and refers to all types of pain, including acute and chronic pain, such as nociceptive pain, e.g. somatic pain and visceral pain; neuropathic pain, e.g., centrally generated pain and peripherally generated pain; and psychogenic pain. The term preferably refers to chronic pain, most preferably nociceptive pain, including somatic pain and visceral pain.

The term “nociceptive pain” is used to include all pain caused by injury to body tissues, including, without limitation, by a cut, bruise, bone fracture, crush injury, burn, and the like. This type of pain is typically aching, sharp, or throbbing. Pain receptors for tissue injury (nociceptor) are located mostly in the skin or in the internal organs.

The term “somatic pain” is used to refer to pain arising from bone, joint, muscle, skin, or connective tissue. This type of pain is typically aching or throbbing in quality and is well localized.

The term “visceral pain” is used herein to refer to pain arising from visceral organs, such as the gastrointestinal tract and pancreas. Visceral pain includes aching and fairly well localized pain caused by tumor involvement of the organ capsule. Another type of visceral pain, which is typically caused by obstruction of hollow viscus, is characterized by intermittent cramping and poorly localized pain.

The term “neuropathic pain” is used herein to refer to pain originating from abnormal processing of sensory input by the peripheral or central nervous system.

By “a disease or condition associated with an increased serum CRP level” is meant any disease or disorder in which the level of serum CRP may be elevated compared to normal controls. Typically a serum CRP level of >3 mg/L is considered elevated. Such diseases and conditions associated with an increased serum CRP level include cardiovascular disease (e.g., coronary artery disease, peripheral artery disease); hypertension; colon cancer; lymphoma; sarcoma; and pancreatitis.

By “sustained release” or “controlled release” is meant that the therapeutically active component (e.g., a corticosteroid) is released from the formulation at a controlled rate such that therapeutically beneficial blood levels (but below toxic levels) of the component are maintained over an extended period of time ranging from e.g., about 2 to about 10 hours, thus, providing, for example, a 2 to 10 hour sustained release dosage form. Whether a pharmaceutical composition is formulated for sustained release, and the range of the sustained release, can be determined by measuring the pharmacokinetic profile of the formulation.

By “delayed release” is meant that the therapeutically active component (e.g., a corticosteroid) is released from the formulation in a delayed manner such that 80%, 85%, 90%, or even 95% of the bioavailable component in the formulation is absorbed into the blood stream of a patient at a delayed time after administration from, e.g., about 2 to about 12 hours, thus, providing, for example, a 2 to 12 hour delayed release dosage form in which at least 80% of the bioavailable therapeutically active component is absorbed between 2 and 12 hours post administration. Whether a pharmaceutical composition is formulated for delayed release, and how long the delay is, can be determined by measuring the pharmacokinetic profile of the formulation.

By “immediate release” is meant that the therapeutically active component (e.g., a corticosteroid) is released from the formulation immediately such that 80%, 85%, 90%, or even 95% of the component in the formulation is absorbed into the blood stream of a patient less than two hours after administration. Whether a pharmaceutical composition is formulated for immediate release can be determined by measuring the pharmacokinetic profile of the formulation.

The term “pharmaceutically acceptable salt” represents those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the compounds of the invention, or separately by reacting the free base function with a suitable organic acid. Representative acid addition salts include acetate, ascorbate, aspartate, benzoate, citrate, digluconate, fumarate, glucoheptonate, glycerophosphate, hemisulfate, heptonate, hexanoate, hydrobromide, hydrochloride, hydroiodide, lactate, malate, maleate, malonate, mesylate, oxalate, phosphate, succinate, sulfate, tartrate, thiocyanate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

Compounds useful in the invention include those described herein in any of their pharmaceutically acceptable forms, including isomers such as diastereomers and enantiomers, salts, esters, amides, thioesters, solvates, and polymorphs thereof, as well as racemic mixtures and pure isomers of the compounds described herein. As an example, by “prednisolone” is meant the free base as well as any pharmaceutically acceptable salt thereof (e.g., prednisolone acetate).

Compounds useful in the invention may also be isotopically labeled compounds. Useful isotopes include hydrogen, carbon, nitrogen, oxygen, phosphorous, fluorine, and chlorine, (e.g., ²H, ³H, ¹³C, ¹⁴C, ¹⁵N, ¹⁸O, ¹⁷O, ³¹P, ³²P, ³⁵S, ¹⁸F, and ³⁶Cl). Isotopically-labeled compounds can be prepared by synthesizing a compound using a readily available isotopically-labeled reagent in place of a non-isotopically-labeled reagent.

Other features and advantages of the invention will be apparent from the following detailed description, the drawings, and the claims.

### DETAILED DESCRIPTION

The invention features methods and compositions for treating a patient diagnosed with, or at risk of developing, musculoskeletal disorders, periodontal disease, or immunoinflammatory disorders by administering a corticosteroid to the patient using a split dose regimen. The invention also features methods and compositions for reducing the serum C-reactive protein (CRP) level in a patient in need thereof, and for treating diseases and conditions associated with an increased serum CRP level.

The invention is described in greater detail below.

### Corticosteroids

Corticosteroids that are useful in the methods, compositions, and kits of this invention are selected from the class of selective glucocorticosteroid receptor agonists (SEGRAs) including, without limitation, 11-alpha, 17-alpha,21-trihydroxypregn-4-ene-3,20-dione; 11-beta, 16-alpha, 17,21-tetrahydroxypregn-4-ene-3,20-dione; 11-beta, 16-alpha, 17,21-tetrahydroxypregn-1,4-diene-3,20-dione; 11-beta, 17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-acetoxypregnenolone; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclomethasone dipropionate; aldosterone; algestone; alphaderm; amadinone; amcinonide; anagestone; androstenedione; anecortave acetate; beclomethasone; beclomethasone dipropionate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; ciclesonide; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortivazol; cortodoxone; daturaolone; deflazacort, 21-deoxycortisol, dehydroepiandrosterone; delmadinone; deoxycorticosterone; deprodone; descinolone; desonide; desoximethasone; dexafen; dexamethas one; dexamethasone 21-acetate; dexamethasone acetate; dexamethasone sodium phosphate; dichlorisone; diflorasone; diflorasone diacetate; diflucortolone; difluprednate; dihydroelatericin a; domoprednate; doxibetasol; ecdysone; ecdysterone; emoxolone; endrysone; enoxolone; fluazacort; flucinolone; flucloronide; fludrocortisone; fludrocortisone acetate; flugestone; flumethasone; flumethasone pivalate; flumoxonide; flunisolide; fluocinolone; fluocinolone acetonide; fluocinonide; fluocortin butyl; 9-fluorocortisone; fluocortolone; fluorohydroxyandrostenedione; fluorometholone; fluorometholone acetate; fluoxymesterone; fluperolone acetate; fluprednidene; fluprednisolone; flurandrenolide; fluticasone; fluticasone propionate; formebolone; formestane; formocortal; gestonorone; glyderinine; halcinonide; halobetasol propionate; halometasone; halopredone; haloprogesterone; hydrocortamate; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; loteprednol etabonate; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednicarbate; prednisolamate; prednisolone; prednisolone 21-diethylaminoacetate; prednisolone 21-hemisuccinate; prednisolone acetate; prednisolone farnesylate; prednisolone hemisuccinate; prednisolone-21(beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone benetonide; triamcinolone diacetate; triamcinolone hexacetonide; trimegestone; turkesterone; and wortmannin.

Standard recommended dosages for various steroid/disease combinations are provided in Table 1, below.

TABLE 1
Standard Recommended Corticosteroid Dosages

| Indication | Route | Drug | Dose | | Schedule |
|---|---|---|---|---|---|
| Psoriasis | oral | prednisolone | 7.5-60 | mg | per day or divided b.i.d. |
| | oral | prednisone | 7.5-60 | mg | per day or divided b.i.d. |
| Asthma | inhaled | beclomethasone dipropionate | 42 | μg/puff) | 4-8 puffs b.i.d. |
| | inhaled | budesonide | (200 | μg/inhalation) | 1-2 inhalations b.i.d. |
| | inhaled | flunisolide | (250 | μg/puff) | 2-4 puffs b.i.d. |
| | inhaled | fluticasone propionate | (44, 110 or 220 | μg/puff) | 2-4 puffs b.i.d. |
| | inhaled | triamcinolone acetonide | (100 | μg/puff) | 2-4 puffs b.i.d. |
| COPD | oral | prednisone | 30-40 | mg | per day |
| Crohn's disease | oral | budesonide | 9 | mg | per day |
| Ulcerative colitis | oral | prednisone | 40-60 | mg | per day |
| | oral | hydrocortisone | 300 | mg (IV) | per day |
| | oral | methylprednisolone | 40-60 | mg | per day |
| Rheumatoid arthritis | oral | prednisone | 10 | mg | per day |

Other standard recommended dosages for corticosteroids are provided, e.g., in the Merck Manual of Diagnosis & Therapy (17th Ed. M H Beers et al., Merck & Co.) and Physicians' Desk Reference 2003 (57^{th} Ed. Medical Economics Staff et al., Medical Economics Co., 2002). In one embodiment, the dosage of corticosteroid administered is a dosage equivalent to a prednisolone dosage, as defined herein. For example, a low dosage of a corticosteroid may be considered as the dosage equivalent to a low dosage of prednisolone. Two or more corticosteroids can be administered in the same treatment.

Equivalent potency in clinical dosing is well known. Information relating to equivalent steroid dosing may be found in the British National Formulary (BNF), 37 Mar. 1999, the content of which is incorporated herein by reference.

The BNF guidelines are included in Table 2 below. More specifically, Table 2 provides doses of steroids equivalent to 5 mg of prednisolone and equivalent to 1 mg of prednisolone when administered in a manner according to this invention.

TABLE 2
Equivalent Dose to Prednisolone

| | | |
|---|---|---|
| | Equal to 5 mg | Equal to 1 mg |
| Drug | prednisolone | prednisolone |

| | | | | |
|---|---|---|---|---|
| betamethasone | 750 | μg | 150 | μg |
| cortisone acetate | 25 | mg | 5 | mg |
| deflazacort | 6 | mg | 1.2 | mg |
| dexamethasone | 750 | μg | 150 | μg |
| hydrocortisone | 20 | mg | 4 | mg |
| methyl prednisone | 4 | mg | 0.8 | mg |
| triamcinolone | 4 | mg | 0.8 | mg |

It is also known (BNF 37 Mar. 1999) from clinical dosing equivalence that doses of triamcinolone, fluticasone and budesonide are broadly similar in nasal administration (110 μg, 100 μg and 200 μg).

### Disease Modifying Anti-Rheumatic Drugs

Disease modifying anti-rheumatic drugs (DMARDs) can be used in the methods, compositions, and kits of the invention. DMARDs are a class of anti-inflammatory drugs. Examples of DMARDs known in the art include, but are not limited to anakinra, auranofin, aurothioglucose, azathioprine, chlorambucil, cyclophosphamide, cyclosporine, D-penicillamine, gold sodium thiomalate (injectable gold), hydroxychloroquine, leflunomide, methotrexate, minocycline, mycophenol mofetil, or sulfasalazine.

Methotrexate is an example of a DMARD that can be used in one embodiment of the combination treatment method of this invention. Methotrexate, also known as Amethopterin, RHEUMATREX® (Lederle Pharmaceutical), or FOLEX® (Aventis), is an antimetabolite that competitively and reversibly inhibits dihydrofolate reductase (DHFR), an enzyme that is part of the folate synthesis metabolic pathway. The affinity of methotrexate for DHFR is about one thousand-fold that of folate for DHFR, which catalyses the conversion of dihydrofolate to the active tetrahydrofolate. Folic acid is needed for the de novo synthesis of the nucleoside thymidine, required for DNA synthesis. Methotrexate is therefore capable of inhibiting the synthesis of DNA, RNA, and thymidylates. Targeting the S-phase of the cell cycle, methotrexate has a greater negative effect on rapidly dividing cells. As a result, methotrexate has been prescribed for treating a number of medical conditions including certain cancers, severe psoriasis, and inflammatory arthritic diseases.

The chemical name for methotrexate is N-[4-[[(2,4-diamino-6-pteridinyl)methyl]methylamino]benzoyl]-L-glutamic acid, although it is commonly present in the form of a sodium salt in pharmaceutical compositions and its amount in such compositions is determined by equivalence to the free acid. Therefore, when a composition is the to contain 10 mg of methotrexate, a greater weight of a sodium salt of methotrexate may be present in the composition. Methotrexate is a generic drug that has been in use for many years and is commercially available through various suppliers. For instance, methotrexate is manufactured and marketed by both Pfizer and Wyeth.

Dosage amounts of DMARDs are known to those skilled in medical arts, and generally range from about 0.1 to 3,000 mg per dose one or more times per week (e.g., 2, 3, 4, 5, 6, or 7 or more times per week), 0.1 to 2,500 mg per dose per week, 0.1 to 2,000 mg per dose per week, 0.1 to 1,000 mg per dose per week, 0.1 to 750 mg per dose per week, 0.1 to 500 mg per dose per week, 0.1 to 250 mg per dose per week, or 0.1 to 100 mg per dose per week.

### Therapy

Therapy according to the invention may be performed alone or in conjunction with another therapy and may be provided at home, the doctor's office, a clinic, a hospital's outpatient department, or a hospital. The duration of the therapy depends on the type of disease or disorder being treated, the age and condition of the patient, the stage and type of the patient's disease, and how the patient responds to the treatment. Additionally, a person having a greater risk of developing musculoskeletal disorders, periodontal disease, or immunoinflammatory disorders may receive treatment to inhibit or delay the onset of symptoms.

In the combination therapies of the invention, the dosage and frequency of administration of each component of the combination can be controlled independently. For example, one compound may be administered three times per day, while the second compound may be administered once per day. Combination therapy may be given in on-and-off cycles that include rest periods so that the patient's body has a chance to recover from any as yet unforeseen side effects. The compounds may also be formulated together such that one administration delivers both compounds.

The compound in question may be administered orally in the form of tablets, capsules, elixirs or syrups, or rectally in the form of suppositories. Parenteral administration of a compound is suitably performed, for example, in the form of saline solutions or with the compound incorporated into liposomes. In cases where the compound in itself is not sufficiently soluble to be dissolved, a solubilizer such as ethanol can be applied.

The methods, compositions, and kits of the invention can include a disease-modifying anti-rheumatic drug (DMARD), such as methotrexate, leflunomide, minocycline, auranofin, gold sodium thiomalate, aurothioglucose, and azathioprine.

Osteoarthritis

The methods, compositions, and kits of the invention may be used for the treatment of osteoarthritis, or pain associated therewith. If desired, one or more agents typically used to treat osteoarthritis may be used with the corticosteroid therapy methods, compositions, and kits of the invention. Such agents include NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), NsIDIs (e.g., cyclosporine, tacrolimus, pimecrolimus, and ISAtx247), or analogs thereof. Thus, in one embodiment, the invention features the combination of any of the foregoing agents and a corticosteroid in the methods and kits for the treatment of osteoarthritis or pain associated therewith.

Chronic Obstructive Pulmonary Disease

In one embodiment, the methods, compositions, and kits of the invention are used for the treatment of chronic obstructive pulmonary disease (COPD). If desired, one or more agents typically used to treat COPD may be used with the corticosteroid therapy methods, compositions, and kits of the invention. Such agents include xanthines (e.g., theophylline), anticholinergic compounds (e.g., ipratropium, tiotropium), biologics, small molecule immunomodulators, and beta receptor agonists/bronchdilators (e.g., ibuterol sulfate, bitolterol mesylate, epinephrine, formoterol fumarate, isoproteronol, levalbuterol hydrochloride, metaproterenol sulfate, pirbuterol scetate, salmeterol xinafoate, and terbutaline. Thus, in one embodiment, the invention features the combination of a bronchodilator and a corticosteroid for treating COPD.

Psoriasis

The methods, compositions, and kits of the invention may be used for the treatment of psoriasis. If desired, one or more antipsoriatic agents typically used to treat psoriasis may be used with the corticosteroid therapy methods, compositions, and kits of the invention. Such agents include biologics (e.g., alefacept, inflixamab, adelimumab, efalizumab, etanercept, and CDP-870), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), vitamin D analogs (e.g., calcipotriene, calcipotriol), psoralens (e.g., methoxsalen), retinoids (e.g., acitretin, tazoretene), DMARDs (e.g., methotrexate), and anthralin. Thus, in one embodiment, the invention features the combination of an antipsoriatic agent and a corticosteroid for treating psoriasis.

Inflammatory Bowel Disease

The methods, compositions, and kits of the invention may be used for the treatment of inflammatory bowel disease. If desired, one or more agents typically used to treat inflammatory bowel disease may be used with the corticosteroid therapy methods, compositions, and kits of the invention. Such agents include biologics (e.g., inflixamab, adelimumab, and CDP-870), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate and azathioprine) and alosetron.

Thus, in one embodiment, the invention features the combination of any of the foregoing agents and a corticosteroid for treating inflammatory bowel disease.

Rheumatoid Arthritis

The methods, compositions, and kits of the invention may be used for the treatment of rheumatoid arthritis. If desired, one or more agents typically used to treat rheumatoid arthritis may be used with the corticosteroid therapy methods, compositions, and kits of the invention. Such agents include NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), COX-2 inhibitors (e.g., rofecoxib, celecoxib, valdecoxib, and lumiracoxib), biologics (e.g., inflixamab, adelimumab, etanercept, CDP-870, rituximab, and atlizumab), small molecule immunomodulators (e.g., VX 702, SCIO 469, doramapimod, RO 30201195, SCIO 323, DPC 333, pranalcasan, mycophenolate, and merimepodib), non-steroidal immunophilin-dependent immunosuppressants (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate, leflunomide, minocycline, auranofin, gold sodium thiomalate, aurothioglucose, and azathioprine), hydroxychloroquine sulfate, and penicillamine. Thus, in one embodiment, the invention features the combination of any of the foregoing agents and a corticosteroid for treating rheumatoid arthritis.

Asthma

The methods, compositions, and kits of the invention may be used for the treatment of asthma. If desired, one or more agents typically used to treat asthma may be used with the corticosteroid therapy methods, compositions, and kits of the invention. Such agents include beta 2 agonists/bronchodilators/leukotriene modifiers (e.g., zafirlukast, montelukast, and zileuton), biologics (e.g., omalizumab), small molecule immunomodulators, anticholinergic compounds, xanthines, ephedrine, guaifenesin, cromolyn sodium, nedocromil sodium, and potassium iodide. Thus, in one embodiment, the invention features the combination of any of the foregoing agents and a corticosteroid for treating asthma.

Pain

The methods, compositions, and kits of the invention may be used for the treatment of pain (e.g., neuropathic pain or nociceptive pain). If desired, one or more agents typically used to treat pain may be used with the corticosteroid therapy methods, compositions, and kits of the invention. Such agents include NSAIDs, opioids, tricyclic antidepressants, anticonvulsants, amantadine, tramadol, oxycodone, buproprion, mexiletine, capsaicin, muscle relaxants, pregabalin, ketamide, analgesics, SSRIs, cannibinoids, sedatives, and anti-anxiety drugs.

Anticonvulsants

The anticonvulsants are used in prevention of the occurrence of epileptic seizures. The goal of an anticonvulsant is to suppress the rapid and excessive firing of neurons that start a seizure. Many anticonvulsants block sodium (Na+) channels, calcium (Ca2+) channels, AMPA receptors, or NMDA receptors. Some anticonvulsants inhibit the metabolism of GABA or increase its release.

Anti-convulsants include barbiturates (e.g., amobarbital, aprobarbital, barbital, butabarbital, butalbital, hexobarbital, methohexital, pentobarbital, secobarbital, sodium thiopental, talbutal, thiobarbital, Phenobarbital, methylphenobarbital, metharbital, barbexaclone), benzodiazepines (e.g., alprazolam, bromazepam, chlordiazepoxide, cinolazepam, clonazepam, clorazepate, diazepam, estazolam, flunitrazepam, flurazepam, halazepam, ketazolam, loprazolam, lorazepam, lormetazepam, medazepam, midazolam, nitrazepam, nordazepam, oxazepam, phenazepam, pinazepam, prazepam, quazepam, temazepam, tetrazepam, and triazolam), carboxamide (e.g., carbamazepine and oxcarbazepine), vigabatrin, progabide, and tiagabine topiramate, gabapentin, pregabalin, hydantoins (e.g., ethotoin, phenyloin, mephenyloin, and fosphenyloin), oxazolidinediones (e.g., paramethadione, trimethadione, ethadione), beclamide, primidone, pyrrolidines (e.g., brivaracetam, levetiracetam, and seletracetam), succinimides (e.g., ethosuximide, phensuximide, and mesuximide), sulfonamides (e.g., acetazolamide, sulthiame, methazolamide, and zonisamide), lamotrigine, pheneturide, phenacemide, valpromide, valnoctamide, and valproate.

Muscle Relaxants

A muscle relaxant is a drug that decreases the tone of a muscle. Muscle relaxants include methocarbamol, baclofen, carisoprodol, chlorzoxazone, cyclobenzaprine, dantrolene, metaxalone, orphenadrine, pancuronium, tizanidine, and dicyclomine.

Analgesics

Analgesics are compounds used to treat pain. Analgesics include opiods (e.g., morphine, codeine, thebaine, oxycodone, hydrocodone, dihydrocodeine, hydromorphone, oxymorphone, nicomorphine, methadone, levo-alphacetylmethadol, fentanyl, alfentanil, sufentanil, remifentanil, ketobemidone, carfentanyl, ohmefentanyl, ketobemidone, allylprodine, prodine, PEPAP, propoxyphene, dextropropoxyphene, dextromoramide, bezitramide, piritramide, pentazocine, phenazocine, buprenorphine, butorphanol, nalbufine, levorphanol, levomethorphan, dezocine, etorphine, lefetamine, tilidine, tramadol, naloxone, and naltrexone), NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), acetaminophen, and COX-2 inhibitors (e.g., rofecoxib, celecoxib, valdecoxib, and lumiracoxib).

Cannibinoids

Cannabinoids are a group of diterpene C21 compounds present in Cannabis sativa L and include a group of substances that are structurally related to THC or that bind to cannabinoid receptors. Cannibinoids include CP-55940, HU-210, SR141716, SR144528, WIN 55, 212-2, JWH-133, Nabilone, Levonantradol, Marinol, and Sativex.

Sedatives

A sedative is a substance that depresses the central nervous system (CNS), resulting in calmness, relaxation, reduction of anxiety, sleepiness, slowed breathing, slurred speech, staggering gait, poor judgment, and slow, uncertain reflexes. Sedatives include chlorpromazine, fluphenazine, haloperidol, loxapine succinate, perphenazine, prochlorperazine, thiothixene, trifluoperazine, clozapine, olanzapine, quetiapine, risperidone, ziprasidone, catnip, Kava Kava, Mandrake, valerian, chloral hydrate, diethyl ether, eszopiclone, ethchlorvynol, ethyl alcohol, gamma-hydroxybutyrate, glutethimide, meprobamate, methaqualone, methyl trichloride, methyprylon, ramelteon, zaleplon, zolpidem, and zopiclone.

Thus, in one embodiment, the invention features the combination of any of the foregoing agents and a corticosteroid for treating pain.

The pain that can be treated using the methods, compositions, and kits of the invention include pain caused as a result of neuropathy, including diabetic neuropathy, polyneuropathy, cancer pain, fibromyalgia, myofascial pain syndrome, osteoarthritis, pancreatic pain, pelvic/perineal pain, post herpetic neuralgia, rheumatoid arthritis, sciatica/lumbar radiculopathy, spinal stenosis, temporo-mandibular joint disorder, HIV pain, trigeminal neuralgia, chronic neuropathic pain, lower back pain, failed back surgery pain, back pain, post-operative pain, post physical trauma pain (including gunshot, road traffic accident, burns), cardiac pain, chest pain, pelvic pain/PID, joint pain (tendonitis, bursitis, acute arthritis), neck pain, bowel pain, phantom limb pain, obstetric pain (labour/C-Section), renal colic, acute herpes zoster pain, acute pancreatitis breakthrough pain (cancer), and dysmenorhoea/endometriosis. The methods, compositions, and kits of the invention can also be used to treat pain caused as a result of inflammatory disease, or as a result of combined inflammatory, autoimmune and neuropathic tissue damage, including rheumatoid arthritis, osteoarthritis, rheumatoid spondylitis, gouty arhritis, and other arthritic conditions, cancer, HIV, chronic pulmonary inflammatory disease, silicosis, pulmonary sarcosis, bone resorption diseases, reperfusion injury (including damage caused to organs as a consequence of reperfusion following ischaemic episodes e.g. myocardial infarcts, strokes), autoimmune damage (including multiple sclerosis, Guillam Barre Syndrome, myasthenia gravis) graft v. host rejection, allograft rejections, fever and myalgia due to infection, AIDS related complex (ARC), keloid formation, scar tissue formation, Crohn's disease, ulcerative colitis and pyresis, irritable bowel syndrome, osteoporosis, cerebral malaria and bacterial meningitis, bowel pain, cancer pain, back pain, fibromyalgia, and post-operative pain.

Periodontal Disease

The methods, compositions, and kits of the invention may be used for the treatment of periodontal disease. If desired, one or more agents typically used to treat periodontal disease may be used with the corticosteroid therapy methods, compositions, and kits of the invention. Such agents include antibiotics (minocycline, penicillin, cephalosporin, tetracycline, oxytetracycline, chlortetracycline, metronidazole, chloramphenicol, streptomycin, neomycin, sulfonamides, phenolic compounds, quarternary ammonium compounds, doxycycline); antiseptics (e.g., chlorhexidine); nonsteroidal antiinflammatories (e.g., flurbiprofen, carprofen, diclofenac, fenbufen, fenclozic acid, fenoprofen, flufenamic acid, ibuprofen, indomethacin, indoprofen, ketoprofen, lonazolac, loxoprofen, meclofenamic acid, mefanamic acid, naproxen, proprionic acids, salicylic acids, sulindac, tolmetin, meloxicam, oxicams, piroxicam, tenoxicam, etodolac, and oxaprozin); tranexamic acid, allantoin; epsilon-aminocaproic acid; lysozyme; dihydrocholesterol; beta-glycyrrhetinic acid; platelet aggregation inhibitors (e.g., abciximab, aspirin, cilostazol, clopidogrel, eptifibatide, ticlopidine, or tirofiban); anticoagulants (e.g., dalteparin, danaparoid, enoxaparin, heparin, tinzaparin, or warfarin); antipyretics (e.g., acetaminophen); ticlopidine; clopidogrel; angiotensin converting enzyme inhibitors; beta blockers; pentoxifylline; cilostazol; estrogen replacement therapy; and lipid-lowering agents (e.g., cholestyramine, colestipol, nicotinic acid, gemfibrozil, probucol, ezetimibe, or statins such as atorvastatin, rosuvastatin, lovastatin simvastatin, pravastatin, cerivastatin, and fluvastatin). These agents may be administered concomitantly or within 14 days of the method of the invention. If desired, one or more of the foregoing agents is coformulated with one or more agents of the invention to form a single composition. Thus, in one embodiment, the invention features one of the foregoing agents and a corticosteroid.

### Cotherapy

If desired, one or more additional agents may be administered in conjunction with the methods, compositions, and kits of the invention. Suitable agents include antibiotics (minocycline, penicillin, cephalosporin, tetracycline, oxytetracycline, chlortetracycline, metronidazole, chloramphenicol, streptomycin, neomycin, sulfonamides, phenolic compounds, quarternary ammonium compounds, doxycycline); antiseptics (e.g., chlorhexidine); nonsteroidal antiinflammatories (e.g., flurbiprofen, carprofen, diclofenac, fenbufen, fenclozic acid, fenoprofen, flufenamic acid, ibuprofen, indomethacin, indoprofen, ketoprofen, lonazolac, loxoprofen, meclofenamic acid, mefanamic acid, naproxen, proprionic acids, salicylic acids, sulindac, tolmetin, meloxicam, oxicams, piroxicam, tenoxicam, etodolac, and oxaprozin); tranexamic acid, allantoin; epsilon-aminocaproic acid; lysozyme; dihydrocholesterol; beta-glycyrrhetinic acid; platelet aggregation inhibitors (e.g., abciximab, aspirin, cilostazol, clopidogrel, eptifibatide, ticlopidine, or tirofiban); anticoagulants (e.g., dalteparin, danaparoid, enoxaparin, heparin, tinzaparin, or warfarin); antipyretics (e.g., acetaminophen); ticlopidine; clopidogrel; angiotensin converting enzyme inhibitors; beta blockers; pentoxifylline; cilostazol; estrogen replacement therapy; and lipid-lowering agents (e.g., cholestyramine, colestipol, nicotinic acid, gemfibrozil, probucol, ezetimibe, or statins such as atorvastatin, rosuvastatin, lovastatin simvastatin, pravastatin, cerivastatin, and fluvastatin). These agents may be administered concomitantly or within 14 days of the method of the invention. If desired, one or more of the foregoing agents is coformulated with one or more agents of the invention to form a single composition. Thus, in one embodiment, the invention features one of the foregoing agents and a corticosteroid.

### Formulation of Compositions

The pharmaceutical compositions of the invention may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Each compound of a combination therapy of the invention may be formulated in a variety of ways that are known in the art. For example, the first and second agents may be formulated together or separately. Desirably, the first and second agents are formulated together for the simultaneous or near simultaneous administration of the agents. The two compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

The individually or separately formulated agents can be packaged together as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions.

The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Corticosteroid Formulations

The formulation of corticosteroids for use in the methods, compositions, and kits of the invention may be as a unit dosage such as a tablet, capsule, ampoule, vial or suspension. A controlled-release formulation may be in matrix, coating, reservoir, osmotic, ion-exchange or density exchange form. It may include a soluble polymer coating which is dissolved or eroded, after administration. Alternatively, there may be an insoluble coating, e.g. of a polymer, through which the active ingredient permeates, as from a reservoir, diffuses, e.g. through a porous matrix, or undergoes osmotic exchange. A further option for a controlled-release formulation involves density exchange, e.g. in the case where the formulation alters on administration, e.g. from microparticles to a gel, so that the active ingredient diffuses or permeates out. Ion-based resins may also be used, the active component being released by ionic exchange, and wherein the rate of release can be controlled by using cationic or anionic forms of the drug. Another type of controlled-release formulation involves pulsed dosing. Further examples of controlled release corticosteroid formulations known in the art are given in PCT Publication No. WO 95/08323 (controlled release containing budesonide); PCT Publication No. WO 91/07172 (oral controlled release glucocorticosteroids for treating ulcerative colitis and Crohn's disease); PCT Publication No. WO 88/00046 (delayed release prednisolone); and U.S. Pat. No. 6,677,326. Other delayed release corticosteroid formulations known in the art include those described in U.S. Pat. No. 6,488,960 (corticosteroid formulations adapted to release 90% by weight of the corticosteroid 2 to 8 hours after administration), and U.S. Pat. No. 5,972,496 (corticosteroid formulations adapted to release 90% by weight of the corticosteroid 1 to 3 hours after administration).

Each dose of the split dose corticosteroid therapy may, independent of each other, be formulated in a variety of ways that are known in the art. The individually or separately formulated pharmaceutical compositions can be packaged together as a kit. Non-limiting examples include kits that contain, e.g., two pills, a pill and a powder, a suppository and a liquid in a vial, or two topical creams. The kit can include optional components that aid in the administration of the unit dose to patients, such as, e.g., vials for reconstituting powder forms, syringes for injection, customized IV delivery systems, or inhalers. Additionally, the unit dose kit can contain instructions for preparation and administration of the compositions.

The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

### Pain, Function, and Fatigue Indices

In order to measure the efficacy of any of the methods, compositions, or kits of the invention, a measurement index may be used. Indices that are useful in the methods, compositions, and kits of the invention include a visual analog scale (VAS), a Likert scale, the Lequesne index, the WOMAC index, the AUSCAN index, the Piper Fatigue Scale, and the Multidimensional Assessment of Fatigue (MAF) scale, each of which is well known in the art. Such indices may be used to measure pain, function, fatigue, stiffness, tenderness, impairment in mobility, soft tissue swelling, bony swelling, or other variables.

A visual analog scale (VAS) provides a measure of a one-dimensional quantity. A VAS generally utilizes a representation of distance, such as a picture of a line with hash marks drawn at regular distance intervals, e.g., ten 1-cm intervals. For example, a patient can be asked to rank a sensation of pain by choosing the spot on the line that best corresponds to the sensation of pain, where one end of the line corresponds to “no pain” (score of 0 cm) and the other end of the line corresponds to “unbearable pain” (score of 10 cm). This procedure provides a simple and rapid approach to obtaining quantitative information about how the patient is experiencing pain. VAS scales can also be used, e.g., to measure fatigue. VAS scales and their use are described, e.g., in U.S. Pat. Nos. 6,709,406 and 6,432,937.

A Likert scale similarly provides a measure of a one-dimensional quantity. Generally, a Likert scale has discrete integer values ranging from a low value (e.g., 0, meaning no pain) to a high value (e.g., 7, meaning extreme pain). A patient experiencing pain is asked to choose a number between the low value and the high value to represent the degree of pain experienced. Likert scales can also be used, e.g., to measure fatigue. Likert scales and their use are described, e.g., in U.S. Pat. Nos. 6,623,040 and 6,766,319.

The Lequesne index and the Western Ontario and McMaster Universities (WOMAC) osteoarthritis index assess pain, function, and stiffness in the knee and hip of OA patients using self-administered questionnaires. Both knee and hip are encompassed by the WOMAC, whereas there is one Lequesne questionnaire for the knee and a separate one for the hip. These questionnaires are useful because they contain more information content in comparison with VAS or Likert. Both the WOMAC index and the Lequesne index questionnaires have been extensively validated in OA, including in surgical settings (e.g., knee and hip arthroplasty). Their metric characteristics do not differ significantly.

The AUSCAN (Australian-Canadian hand arthritis) index employs a valid, reliable, and responsive patient self-reported questionnaire. In one instance, this questionnaire contains 15 questions within three dimensions (Pain, 5 questions; Stiffness, 1 question; and Physical function, 9 questions). An AUSCAN index may utilize, e.g., a Likert or a VAS scale.

The Piper Fatigue scale is a 41-item measure of fatigue developed for research purposes and tested with oncology patients (Piper et al. (1989), The development of an instrument to measure the subjective dimension of fatigue. In S. Funk, E. Tornquist, M. Champagne, & R. Wiese (Eds.). Key aspects of comfort: Management of pain, fatigue, and nausea (pp. 199-207). New York: Springer.) The Multidimensional Assessment of Fatigue (MAF) scale, a revision of the Piper Fatigue scale, contains 15 items and measures four dimensions of fatigue: severity (#1-2), distress (#3), degree of interference in activities of daily living (#4-14), and frequency (#15), with scores ranging from 1 (no fatigue) to 50 (severe fatigue). The MAF has been validated in RA patients (Belza, J. Rheumatol. 22:639-643, 1995).

### Rheumatoid Arthritis Indices

In order to measure the efficacy of any of the methods, compositions, or kits of the invention, a measurement index may be used. Indices that are useful in the methods, compositions, and kits of the invention include the ACR-20/50/70 and the disease activity score (DAS).

ACR-20/50/70

ACR-20/50/70 is a widely accepted composite index of improvement in RA proposed by the American College of Rheumatology (ACR). ACR-20/50/70 refers to a composite improvement of 20%, 50% or 70% in swollen joint count, tender joint count, and 3 or more of the following 5 measures: patient's own global assessment of RA disease activity; physician's global assessment of disease activity; patient's own assessment of pain due to RA; acute-phase reactant (CRP); and patient's self-addressed disability (Health Assessment Questionnaire).

DAS28

The disease activity score (DAS) is a combined index that was developed in Nijmegen in the 1980s to measure the disease activity in patients with RA. It has been extensively validated for its use in clinical trials in combination with the European League Against Rheumatism (EULAR) response criteria. To calculate the DAS28, the number of swollen joints and tender joints should be assessed using 28-joint counts, the CRP levels should be measured in mg/L, and the patients general health (GH) or global disease activity measured on a Visual Analog Scale (VAS) of 100 mm must be obtained. Using this data, the DAS28 using CRP (mg/L) can be calculated using the following formula:

DAS28=0.56*sqrt(tender28)+0.28*sqrt(swollen28)+0.36*ln(CRP+1)+0.014*GH+0.96

The DAS28 provides a number between 0 and 10 indicating the current activity of RA in the patient. A DAS above 5.1 means high disease activity, and below 3.2 indicates low activity. Remission is achieved by a DAS28 lower than 2.6.

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

### EXAMPLES

### Study Protocol

We conducted a 14 week blinded, randomized study including a split dose corticosteroid therapy or placebo, with regular CRP and inflammatory cytokine measurements. The study population had active rheumatoid arthritis. The subject must otherwise have been in good general health.

During the study, subjects attended the following study visits:

Screening Visit
Day 1 (Baseline Visit)—Start of Run-In Period
Day 14 (±2 days)—End of Run-In Period/Start of Treatment Period
Day 42 (±2 days)
Day 70 (±2 days)—End of Treatment Period/Start of Withdrawal Period
Day 98 (±2 days)—End of Study

All eligible subjects received DMARD therapy in a stable dose. Subjects were evaluated for study eligibility at the Screening visit, which was conducted within 14 days before the first dose of study drug. The subject provided written informed consent to participate in the study before any Screening laboratory samples were collected or evaluations performed.

All subjects were given 3 mg prednisolone alone for 2 weeks (Run-In Period). Subjects were then randomized into treatment groups to additionally receive 10 mg paroxetine, 20 mg paroxetine or placebo tablets for 8 weeks (Combination Treatment Period). The powered study ended after these first 10 weeks. All subjects continued in an un-powered part of the study for an additional 4 weeks (Withdrawal Period). In this portion of the study, half of the subjects on 10 mg or 20 mg paroxetine plus 3 mg prednisolone stopped taking paroxetine, and instead took 3 mg prednisolone plus placebo, while the other half stopped taking prednisolone, and instead took either dose of paroxetine plus placebo. Similarly, half of the subjects on prednisolone plus placebo stopped the prednisolone, and instead took two placebo tablets, while the other half continued the regimen of 3 mg prednisolone plus placebo. A schematic of the study design is shown below.

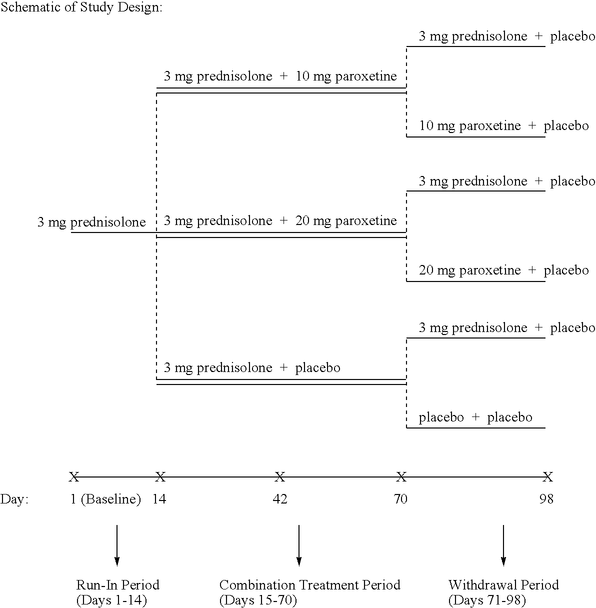

The drugs were blister packed as follows:

Steroid Run-In Period (Days 1-14)

| | 8 am | 1 pm |
|---|---|---|
| All subjects | 1 mg prednisolone | 1 mg prednisolone |
| | | 1 mg prednisolone |

Combination Treatment Period (Days 15-70)

| | 8 am | 1 pm |
|---|---|---|
| Dose Level 1 | 10 mg paroxetine | 1 mg prednisolone |
| | 1 mg prednisolone | 1 mg prednisolone |
| Dose Level 2 | 20 mg paroxetine | 1 mg prednisolone |
| | 1 mg prednisolone | 1 mg prednisolone |
| Placebo | placebo | 1 mg prednisolone |
| | 1 mg prednisolone | 1 mg prednisolone |

Withdrawal Period (Days 71-98)

| | 8 am | 1 pm |
|---|---|---|
| Dose Level 1a | placebo | 1 mg prednisolone |
| | 1 mg prednisolone | 1 mg prednisolone |
| Dose Level 1b | 10 mg paroxetine | placebo |
| | placebo | placebo |
| Dose Level 2a | placebo | 1 mg prednisolone |
| | 1 mg prednisolone | 1 mg prednisolone |
| Dose Level 2b | 20 mg paroxetine | placebo |
| | placebo | placebo |
| Placebo A | placebo | 1 mg prednisolone |
| | 1 mg prednisolone | 1 mg prednisolone |
| Placebo B | placebo | placebo |
| | placebo | placebo |

The results of the study are shown in Tables 3-10. While the paroxetine/prednisolone combinations weres not statistically significant when compared to 3 mg prednisolone as measured by ACR20 at day 70, these combinations did achieve statistical significant when compared to 3 mg prednisolone alone as measured by ACR20 and ACR50 at earlier time points.

### Study Results

TABLE 3
Summary of American College of Rheumatology (ACR) Responses for
Prednisolone Vs. Combined Paroxetine Groups
ITT Population (Missing Values = Non-Responder)

| | | | 3 mg | |
|---|---|---|---|---|
| | | Response | Prednisolone | 3 mg Prednisolone |
| | | from | Placebo | 10-20 mg Paroxetine |
| ACR | Visit | Baseline | (N = 69) | (N = 140) |
| ACR20 | Day 14 | Yes | 16 (23) | 32 (23) |
| | | No | 53 (77) | 108 (77) |
| | | Pvalue | | 0.9573 |
| | Day 42 | Yes | 25 (36) | 73 (52) |
| | | No | 44 (64) | 67 (48) |
| | | Pvalue | | 0.0302 |
| | Day 70 | Yes | 30 (43) | 58 (41) |
| | | No | 39 (57) | 82 (59) |
| | | Pvalue | | 0.7778 |
| ACR50 | Day 14 | Yes | 5 (7) | 5 (4) |
| | | No | 64 (93) | 135 (96) |
| | | Pvalue | | 0.3036* |
| | Day 42 | Yes | 9 (13) | 31 (22) |
| | | No | 60 (87) | 109 (78) |
| | | Pvalue | | 0.1158 |
| | Day 70 | Yes | 14 (20) | 30 (21) |
| | | No | 55 (80) | 110 (79) |
| | | Pvalue | | 0.8494 |
| ACR70 | Day 14 | Yes | 3 (4) | 0 |
| | | No | 66 (96) | 140 (100) |
| | | Pvalue | | 0.0349* |
| | Day 42 | Yes | 2 (3) | 10 (7) |
| | | No | 67 (97) | 130 (93) |
| | | Pvalue | | 0.3442* |
| | Day 70 | Yes | 4 (6) | 10 (7) |
| | | No | 65 (94) | 130 (93) |
| | | Pvalue | | 1.0000* |

Note:
p-value derived from a Chi-Square Test determining differences in proportions between treatment group and placebo.
*If any expected cell count is less than 5 Fisher Exact test used.

TABLE 4
Summary of American College of Rheumatology (ACR) Responses
for Prednisolone Vs. Each Paroxetine Group
ITT Population (Missing Values = Non-Responder)

| | | | 3 mg Prednisolone | 3 mg Prednisolone | 3 mg Prednisolone |
|---|---|---|---|---|---|
| | | Response | Placebo | 10 mg Paroxetine | 20 mg Paroxetine |
| ACR | Visit | from Baseline | (N = 69) | (N = 71) | (N = 69) |
| ACR20 | Day 14 | Yes | 16 (23) | 14 (20) | 18 (26) |
| | | No | 53 (77) | 57 (80) | 51 (74) |
| | | Pvalue | | 0.6169 | 0.6928 |
| | Day 42 | Yes | 25 (36) | 35 (49) | 38 (55) |
| | | No | 44 (64) | 36 (51) | 31 (45) |
| | | Pvalue | | 0.1184 | 0.0263 |
| | Day 70 | Yes | 30 (43) | 27 (38) | 31 (45) |
| | | No | 39 (57) | 44 (62) | 38 (55) |
| | | Pvalue | | 0.5117 | 0.8639 |
| ACR50 | Day 14 | Yes | 5 (7) | 3 (4) | 2 (3) |
| | | No | 64 (93) | 68 (96) | 67 (97) |
| | | Pvalue | | 0.4902* | 0.4409* |
| | Day 42 | Yes | 9 (13) | 9 (13) | 22 (32) |
| | | No | 60 (87) | 62 (87) | 47 (68) |
| | | Pvalue | | 0.9482 | 0.0080 |
| | Day 70 | Yes | 14 (20) | 11 (15) | 19 (28) |
| | | No | 55 (80) | 60 (85) | 50 (72) |
| | | Pvalue | | 0.4588 | 0.3184 |
| ACR70 | Day 14 | Yes | 3 (4) | 0 | 0 |
| | | No | 66 (96) | 71 (100) | 69 (100) |
| | | Pvalue | | 0.1171* | 0.2445* |
| | Day 42 | Yes | 2 (3) | 3 (4) | 7 (10) |
| | | No | 67 (97) | 68 (96) | 62 (90) |
| | | Pvalue | | 1.0000* | 0.1652* |
| | Day 70 | Yes | 4 (6) | 4 (6) | 6 (9) |
| | | No | 65 (94) | 67 (94) | 63 (91) |
| | | Pvalue | | 1.0000* | 0.5114 |

TABLE 5
Summary of CRP for Prednisolone Vs. Combined Paroxetine Groups
ITT Population (LOCF)

| | 3 mg Prednisolone + Placebo | | | 3 mg Prednisolone + 10-20 mg Paroxetine | | |
|---|---|---|---|---|---|---|
| | (N = 69) | | | (N = 140) | | |
| | | Change from | % Change from | | Change from | % Change from |
| Visit | Value | baseline | baseline | Value | baseline | baseline |

Baseline

| | | |
|---|---|---|
| N | 69 | 140 |
| Mean (Std Dev) | 18.83 (23.749) | 12.39 (15.780) |
| Median | 8.90 | 6.55 |
| Min, Max | 0.3, 135.3 | 0.2, 91.6 |

Day 14

| | | | | | | |
|---|---|---|---|---|---|---|
| N | 69 | 69 | 69 | 140 | 140 | 140 |
| Mean (Std Dev) | 11.42 (17.413) | −7.40 (20.149) | 7.11 (215.970) | 8.36 (11.624) | −4.03 (9.748) | −16.96 (80.108) |
| Median | 6.20 | −1.70 | −32.26 | 4.45 | −1.45 | −36.28 |
| Min, Max | 0.3, 107.3 | −83.2, 77.9 | −95.8, 1700.0 | 0.0, 70.0 | −63.8, 29.0 | −100.0, 522.2 |
| p-value | | | | | 0.6263 | 0.4161 |

Day 42

| | | | | | | |
|---|---|---|---|---|---|---|
| N | 69 | 69 | 69 | 140 | 140 | 140 |
| Mean (Std Dev) | 10.22 (12.443) | −8.61 (18.009) | −17.67 (58.589) | 9.89 (14.263) | −2.50 (9.573) | 3.67 (209.843) |
| Median | 5.40 | −1.00 | −23.53 | 4.25 | −1.05 | −28.11 |
| Min, Max | 0.3, 70.6 | −81.4, 21.5 | −92.5, 187.3 | 0.1, 72.0 | −35.8, 55.3 | −97.6, 2340.0 |
| p-value | | | | | 0.2687 | 0.7120 |

Day 70

| | | | | | | |
|---|---|---|---|---|---|---|
| N | 69 | 69 | 69 | 140 | 140 | 140 |
| Mean (Std Dev) | 9.88 (11.956) | −8.95 (18.996) | −13.66 (64.905) | 10.62 (14.625) | −1.77 (8.443) | 6.02 (106.063) |
| Median | 5.70 | −1.20 | −29.27 | 4.00 | −0.80 | −15.47 |
| Min, Max | 0.2, 64.6 | −87.7, 15.5 | −92.8, 187.3 | 0.2, 81.0 | −37.9, 29.1 | −99.0, 740.0 |
| p-value | | | | | 0.1504 | 0.2078 |

Note:
p-value derived from a Wilcoxon Rank Sum Test comparing differences in distributions of changes (% Changes) between treatment group and placebo.

TABLE 6
Summary of CRP for Prednisolone Vs. 10 mg Paroxetine
ITT Population (LOCF)

| | 3 mg Prednisolone + Placebo | | | 3 mg Prednisolone + 10 mg Paroxetine | | |
|---|---|---|---|---|---|---|
| | (N = 69) | | | (N = 71) | | |
| | | Change from | % Change from | | Change from | % Change from |
| Visit | Value | baseline | baseline | Value | baseline | baseline |

Baseline

| | | |
|---|---|---|
| N | 69 | 71 |
| Mean (Std Dev) | 18.83 (23.749) | 10.92 (13.321) |
| Median | 8.90 | 6.50 |
| Min, Max | 0.3, 135.3 | 0.2, 58.7 |

Day 14

| | | | | | | |
|---|---|---|---|---|---|---|
| N | 69 | 69 | 69 | 71 | 71 | 71 |
| Mean (Std Dev) | 11.42 (17.413) | −7.40 (20.149) | 7.11 (215.970) | 7.74 (10.712) | −3.18 (7.126) | −15.70 (71.753) |
| Median | 6.20 | −1.70 | −32.26 | 4.40 | −1.00 | −34.88 |
| Min, Max | 0.3, 107.3 | −83.2, 77.9 | −95.8, 1700.0 | 0.0, 49.7 | −27.0, 19.2 | −100.0, 316.0 |
| p-value | | | | | 0.4456 | 0.7128 |

Day 42

| | | | | | | |
|---|---|---|---|---|---|---|
| N | 69 | 69 | 69 | 71 | 71 | 71 |
| Mean (Std Dev) | 10.22 (12.443) | −8.61 (18.009) | −17.67 (58.589) | 9.54 (14.300) | −1.38 (10.398) | 27.51 (288.446) |
| Median | 5.40 | −1.00 | −23.53 | 4.20 | −0.80 | −28.80 |
| Min, Max | 0.3, 70.6 | −81.4, 21.5 | −92.5, 187.3 | 0.2, 72.0 | −35.8, 55.3 | −86.3, 2340.0 |
| p-value | | | | | 0.1384 | 0.3673 |

Day 70

| | | | | | | |
|---|---|---|---|---|---|---|
| N | 69 | 69 | 69 | 71 | 71 | 71 |
| Mean (Std Dev) | 9.88 (11.956) | −8.95 (18.996) | −13.66 (64.905) | 9.82 (14.480) | −1.09 (7.437) | 16.45 (120.350) |
| Median | 5.70 | −1.20 | −29.27 | 3.90 | −0.60 | −15.38 |
| Min, Max | 0.2, 64.6 | −87.7, 15.5 | −92.8, 187.3 | 0.2, 81.0 | −22.4, 29.1 | −87.5, 740.0 |
| p-value | | | | | 0.0753 | 0.1072 |

Note:
p-value derived from a Wilcoxon Rank Sum Test comparing differences in distributions of changes (% Changes) between treatment group and placebo.

TABLE 7
Summary of CRP for Prednisolone Vs. 20 mg Paroxetine
ITT Population (LOCF)

| | 3 mg Prednisolone + Placebo | | | 3 mg Prednisolone + 20 mg Paroxetine | | |
|---|---|---|---|---|---|---|
| | (N = 69) | | | (N = 69) | | |
| | | Change from | % Change from | | Change from | % Change from |
| Visit | Value | baseline | baseline | Value | baseline | baseline |

Baseline

| | | |
|---|---|---|
| N | 69 | 69 |
| Mean (Std Dev) | 18.83 (23.749) | 13.90 (17.938) |
| Median | 8.90 | 6.80 |
| Min, Max | 0.3, 135.3 | 0.7, 91.6 |

Day 14

| | | | | | | |
|---|---|---|---|---|---|---|
| N | 69 | 69 | 69 | 69 | 69 | 69 |
| Mean (Std Dev) | 11.42 (17.413) | −7.40 (20.149) | 7.11 (215.970) | 9.00 (12.540) | −4.90 (11.850) | −18.26 (88.400) |
| Median | 6.20 | −1.70 | −32.26 | 4.60 | −1.70 | −41.28 |
| Min, Max | 0.3, 107.3 | −83.2, 77.9 | −95.8, 1700.0 | 0.3, 70.0 | −63.8, 29.0 | −85.2, 522.2 |
| p-value | | | | | 0.9441 | 0.2967 |

Day 42

| | | | | | | |
|---|---|---|---|---|---|---|
| N | 69 | 69 | 69 | 69 | 69 | 69 |
| Mean (Std Dev) | 10.22 (12.443) | −8.61 (18.009) | −17.67 (58.589) | 10.26 (14.320) | −3.64 (8.566) | −20.86 (56.199) |
| Median | 5.40 | −1.00 | −23.53 | 4.50 | −1.20 | −27.42 |
| Min, Max | 0.3, 70.6 | −81.4, 21.5 | −92.5, 187.3 | 0.1, 64.0 | −31.9, 17.5 | −97.6, 202.1 |
| p-value | | | | | 0.6771 | 0.7840 |

Day 70

| | | | | | | |
|---|---|---|---|---|---|---|
| N | 69 | 69 | 69 | 69 | 69 | 69 |
| Mean (Std Dev) | 9.88 (11.956) | −8.95 (18.996) | −13.66 (64.905) | 11.44 (14.834) | −2.46 (9.372) | −4.72 (88.625) |
| Median | 5.70 | −1.20 | −29.27 | 5.30 | −1.10 | −16.67 |
| Min, Max | 0.2, 64.6 | −87.7, 15.5 | −92.8, 187.3 | 0.2, 64.0 | −37.9, 25.3 | −99.0, 482.5 |
| p-value | | | | | 0.4888 | 0.5808 |

Note:
p-value derived from a Wilcoxon Rank Sum Test comparing differences in distributions of changes (% Changes) between treatment group and placebo.

TABLE 8
Summary of Patient Pain Assessment (VAS) Scores for Prednisolone Vs. Combined Paroxetine Groups
ITT Population (LOCF)

| | 3 mg Prednisolone + Placebo | | | 3 mg Prednisolone + 10-20 mg Paroxetine | | |
|---|---|---|---|---|---|---|
| | (N = 69) | | | (N = 140) | | |
| | | Change from | % Change from | | Change from | % Change from |
| Visit | Value | baseline | baseline | Value | baseline | baseline |

Baseline

| | | |
|---|---|---|
| N | 69 | 140 |
| Mean (Std Dev) | 53.31 (22.493) | 52.31 (22.861) |
| Median | 52.00 | 49.00 |
| Min, Max | 7.0, 97.0 | 7.0, 98.0 |

Day 14

| | | | | | | |
|---|---|---|---|---|---|---|
| N | 69 | 69 | 69 | 140 | 140 | 140 |
| Mean (Std Dev) | 45.58 (25.283) | −7.73 (19.337) | −13.65 (38.137) | 41.85 (23.243) | −10.46 (21.686) | −14.56 (45.212) |
| Median | 47.00 | −5.00 | −11.11 | 40.50 | −7.00 | −13.21 |
| Min, Max | 2.0, 97.0 | −81.0, 35.0 | −94.7, 106.7 | 1.0, 93.0 | −79.0, 40.0 | −94.4, 205.6 |
| p-value | | | | | 0.3842 | 0.5690 |

Day 42

| | | | | | | |
|---|---|---|---|---|---|---|
| N | 69 | 69 | 69 | 140 | 140 | 140 |
| Mean (Std Dev) | 42.74 (26.143) | −10.57 (21.630) | −19.23 (47.955) | 33.37 (25.041) | −18.94 (22.540) | −33.53 (47.151) |
| Median | 37.00 | −9.00 | −23.44 | 27.00 | −18.00 | −37.26 |
| Min, Max | 2.0, 97.0 | −64.0, 56.0 | −93.5, 186.7 | 0.0, 97.0 | −85.0, 36.0 | −100.0, 163.2 |
| p-value | | | | | 0.0206 | 0.0200 |

Day 70

| | | | | | | |
|---|---|---|---|---|---|---|
| N | 69 | 69 | 69 | 140 | 140 | 140 |
| Mean (Std Dev) | 41.04 (25.466) | −12.28 (23.864) | −17.84 (57.121) | 37.10 (27.863) | −15.21 (27.066) | −22.72 (68.982) |
| Median | 39.00 | −10.00 | −25.64 | 31.00 | −13.00 | −29.11 |
| Min, Max | 2.0, 97.0 | −69.0, 49.0 | −90.5, 233.3 | 0.0, 97.0 | −91.0, 70.0 | −100.0, 466.7 |
| p-value | | | | | 0.4716 | 0.3293 |

Note:
p-value derived from a Wilcoxon Rank Sum Test comparing differences in distributions of change (% Changes) between treatment group and placebo.

TABLE 9
Summary of Patient Pain Assessment (VAS) Scores for Prednisolone Vs. 10 mg Paroxetine
ITT Population (LOCF)

| | 3 mg Prednisolone + Placebo | | | 3 mg Prednisolone + 10 mg Paroxetine | | |
|---|---|---|---|---|---|---|
| | (N = 69) | | | (N = 71) | | |
| | | Change from | % Change from | | Change from | % Change from |
| Visit | Value | baseline | baseline | Value | baseline | baseline |

Baseline

| | | |
|---|---|---|
| N | 69 | 71 |
| Mean (Std Dev) | 53.31 (22.493) | 51.05 (20.405) |
| Median | 52.00 | 50.00 |
| Min, Max | 7.0, 97.0 | 9.0, 93.0 |

Day 14

| | | | | | | |
|---|---|---|---|---|---|---|
| N | 69 | 69 | 69 | 71 | 71 | 71 |
| Mean (Std Dev) | 45.58 (25.283) | −7.73 (19.337) | −13.65 (38.137) | 45.15 (21.788) | −5.89 (19.259) | −4.34 (48.497) |
| Median | 47.00 | −5.00 | −11.11 | 46.00 | −3.00 | −4.35 |
| Min, Max | 2.0, 97.0 | −81.0, 35.0 | −94.7, 106.7 | 3.0, 93.0 | −42.0, 40.0 | −93.2, 205.6 |
| p-value | | | | | 0.6820 | 0.4165 |

Day 42

| | | | | | | |
|---|---|---|---|---|---|---|
| N | 69 | 69 | 69 | 71 | 71 | 71 |
| Mean (Std Dev) | 42.74 (26.143) | −10.57 (21.630) | −19.23 (47.955) | 35.84 (24.433) | −15.21 (20.510) | −27.16 (47.385) |
| Median | 37.00 | −9.00 | −23.44 | 29.00 | −14.00 | −28.57 |
| Min, Max | 2.0, 97.0 | −64.0, 56.0 | −93.5, 186.7 | 0.0, 97.0 | −63.0, 36.0 | −100.0, 163.2 |
| p-value | | | | | 0.2255 | 0.3060 |

Day 70

| | | | | | | |
|---|---|---|---|---|---|---|
| N | 69 | 69 | 69 | 71 | 71 | 71 |
| Mean (Std Dev) | 41.04 (25.466) | −12.28 (23.864) | −17.84 (57.121) | 39.35 (28.086) | −11.70 (24.235) | −20.92 (51.283) |
| Median | 39.00 | −10.00 | −25.64 | 32.00 | −13.00 | −24.62 |
| Min, Max | 2.0, 97.0 | −69.0, 49.0 | −90.5, 233.3 | 0.0, 97.0 | −65.0, 50.0 | −100.0, 119.0 |
| p-value | | | | | 0.9502 | 0.9453 |

Note:
p-value derived from a Wilcoxon Rank Sum Test comparing differences in distributions of changes (% Changes) between treatment group and placebo.

TABLE 10
Summary of Patient Pain Assessment (VAS) Scores for Prednisolone Vs. 20 mg Paroxetine
ITT Population (LOCF)

| | 3 mg Prednisolone + Placebo | | | 3 mg Prednisolone + 20 mg Paroxetine | | |
|---|---|---|---|---|---|---|
| | (N = 69) | | | (N = 69) | | |
| | | Change from | % Change from | | Change from | % Change from |
| Visit | Value | baseline | baseline | Value | baseline | baseline |

Baseline

| | | |
|---|---|---|
| N | 69 | 69 |
| Mean (Std Dev) | 53.31 (22.493) | 53.61 (25.226) |
| Median | 52.00 | 49.00 |
| Min, Max | 7.0, 97.0 | 7.0, 98.0 |

Day 14

| | | | | | | |
|---|---|---|---|---|---|---|
| N | 69 | 69 | 69 | 69 | 69 | 69 |
| Mean (Std Dev) | 45.58 (25.283) | −7.73 (19.337) | −13.65 (38.137) | 38.46 (24.342) | −15.15 (23.139) | −25.07 (39.195) |
| Median | 47.00 | −5.00 | −11.11 | 36.00 | −10.00 | −24.62 |
| Min, Max | 2.0, 97.0 | −81.0, 35.0 | −94.7, 106.7 | 1.0, 88.0 | −79.0, 34.0 | −94.4, 63.0 |
| p-value | | | | | 0.0534 | 0.0689 |

Day 42

| | | | | | | |
|---|---|---|---|---|---|---|
| N | 69 | 69 | 69 | 69 | 69 | 69 |
| Mean (Std Dev) | 42.74 (26.143) | −10.57 (21.630) | −19.23 (47.955) | 30.83 (25.580) | −22.78 (24.001) | −40.08 (46.338) |
| Median | 37.00 | −9.00 | −23.44 | 23.00 | −20.00 | −49.38 |
| Min, Max | 2.0, 97.0 | −64.0, 56.0 | −93.5, 186.7 | 0.0, 87.0 | −85.0, 26.0 | −100.0, 118.2 |
| p-value | | | | | 0.0052 | 0.0027 |

Day 70

| | | | | | | |
|---|---|---|---|---|---|---|
| N | 69 | 69 | 69 | 69 | 69 | 69 |
| Mean (Std Dev) | 41.04 (25.466) | −12.28 (23.864) | −17.84 (57.121) | 34.78 (27.643) | −18.83 (29.440) | −24.58 (83.742) |
| Median | 39.00 | −10.00 | −25.64 | 29.50 | −13.00 | −40.38 |
| Min, Max | 2.0, 97.0 | −69.0, 49.0 | −90.5, 233.3 | 0.0, 92.0 | −91.0, 70.0 | −100.0, 466.7 |
| p-value | | | | | 0.1861 | 0.1034 |

### OTHER EMBODIMENTS

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method for treating an immunoinflammatory disorder in a patient in need thereof, said method comprising administering to said patient a first dose of a first corticosteroid at a time T₁ followed by a second dose of a second corticosteroid at a time T₂, wherein said time T₁ and said time T₂ are separated by 2 to 10 hours and wherein said first corticosteroid and said second corticosteroid are administered in amounts that together are sufficient to treat said patient.

2. A method for treating an immunoinflammatory disorder in a patient in need thereof, said method comprising administering to said patient a pharmaceutical composition in unit dosage form comprising a first dose of a first corticosteroid formulated for immediate release and a second dose of a second corticosteroid formulated for 2 to 10 hour delayed release, wherein said first dose and said second dose are administered in amounts that together are sufficient to treat said patient.

3. A method for treating an immunoinflammatory disorder in a patient in need thereof, said method comprising administering to said patient a pharmaceutical composition in unit dosage form comprising a corticosteroid formulated for 2 to 10 hour sustained release, wherein said corticosteroid is from 1 to 30 mg of prednisolone, or an equivalent, equipotent amount of another corticosteroid.

4. The method of any of claims 1-3, wherein said immunoinflammatory disorder is rheumatoid arthritis, Crohn's disease, ulcerative colitis, asthma, chronic obstructive pulmonary disease, polymylagia rheumatica, giant cell arteritis, systemic lupus erythematosus, atopic dermatitis, multiple sclerosis, myasthenia gravis, psoriasis, ankylosing spondylitis, cirrhosis, or psoriatic arthritis.

5. A method for treating a disease or condition associated with an increased serum CRP level in a patient in need thereof, said method comprising administering to said patient a first dose of a first corticosteroid at a time T₁ followed by a second dose of a second corticosteroid at a time T₂, wherein said time T₁ and said time T₂ are separated by 2 to 10 hours and wherein said first dose and said second dose are administered in amounts that together are sufficient to treat said patient.

6. A method for treating a disease or condition associated with an increased serum CRP level in a patient in need thereof, said method comprising administering to said patient a pharmaceutical composition in unit dosage form comprising a first dose of a first corticosteroid formulated for immediate release and a second dose of a second corticosteroid formulated for 2 to 10 hour delayed release, wherein said first dose and said second dose are administered in amounts that together are sufficient to treat said patient.

7. A method for treating a disease or condition associated with an increased serum CRP level in a patient in need thereof, said method comprising administering to said patient a pharmaceutical composition in unit dosage form comprising a corticosteroid formulated for 2 to 10 hour sustained release, wherein said corticosteroid is from 1 to 30 mg of prednisolone, or an equivalent, equipotent amount of another corticosteroid.

8. The method of any of claims 5-7, wherein said disease or condition associated with an increased serum CRP level is selected from cardiovascular disease, hypertension, colon cancer, lymphoma, and sarcoma.

9. A method for reducing the serum C-reactive protein (CRP) level in a patient in need thereof, said method comprising administering to said patient a first dose of a first corticosteroid at a time T₁ followed by a second dose of a second corticosteroid at a time T₂, wherein said time T₁ and said time T₂ are separated by 2 to 10 hours and wherein said first dose and said second dose are administered in amounts that together are sufficient to reduce the serum CRP level in said patient.

10. A method for reducing the serum C-reactive protein (CRP) level in a patient in need thereof, said method comprising administering to said patient a pharmaceutical composition in unit dosage form comprising a first dose of a first corticosteroid formulated for immediate release and a second dose of a second corticosteroid formulated for 2 to 10 hour delayed release, wherein said first dose and said second dose are administered in amounts that together are sufficient to treat said patient.

11. A method for reducing the serum C-reactive protein (CRP) level in a patient in need thereof, said method comprising administering to said patient a pharmaceutical composition in unit dosage form comprising a corticosteroid formulated for 2 to 10 hour sustained release, wherein said corticosteroid is from 1 to 30 mg of prednisolone, or an equivalent, equipotent amount of another corticosteroid.

12. A method for treating pain or fatigue associated with a musculoskeletal disorder in a patient in need thereof, said method comprising administering to said patient a first dose of a first corticosteroid at a time T₁ followed by a second dose of a second corticosteroid at a time T₂, wherein said time T₁ and said time T₂ are separated by 2 to 10 hours and wherein said first dose of a corticosteroid and said second dose of a corticosteroid are administered in amounts that together are sufficient to treat said patient.

13. A method for treating pain or fatigue associated with a musculoskeletal disorder in a patient in need thereof, said method comprising administering to said patient a pharmaceutical composition in unit dosage form comprising a first dose of a first corticosteroid formulated for immediate release and a second dose of a second corticosteroid formulated for 2 to 10 hour delayed release, wherein said first dose and said second dose are administered in amounts that together are sufficient to treat said patient.

14. A method for treating pain or fatigue associated with a musculoskeletal disorder in a patient in need thereof, said method comprising administering to said patient a pharmaceutical composition in unit dosage form comprising a corticosteroid formulated for 2 to 10 hour sustained release, wherein said corticosteroid is from 1 to 30 mg of prednisolone, or an equivalent, equipotent amount of another corticosteroid.

15. A method for treating tenderness, impairment in mobility, soft tissue swelling, or bony swelling associated with a musculoskeletal disorder in a patient in need thereof, said method comprising administering to said patient a first dose of a first corticosteroid at a time T₁ followed by a second dose of a second corticosteroid at a time T₂, wherein said time T₁ and said time T₂ are separated by 2 to 10 hours and wherein said first dose of a corticosteroid and said second dose of a corticosteroid are administered in amounts that together are sufficient to treat said patient.

16. A method for treating tenderness, impairment in mobility, soft tissue swelling, or bony swelling associated with a musculoskeletal disorder in a patient in need thereof, said method comprising administering to said patient a pharmaceutical composition in unit dosage form comprising a first dose of a first corticosteroid formulated for immediate release and a second dose of a second corticosteroid formulated for 2 to 10 hour delayed release, wherein said first dose and said second dose are administered in amounts that together are sufficient to treat said patient.

17. A method for treating tenderness, impairment in mobility, soft tissue swelling, or bony swelling associated with a musculoskeletal disorder in a patient in need thereof, said method comprising administering to said patient a pharmaceutical composition in unit dosage form comprising a corticosteroid formulated for 2 to 10 hour sustained release, wherein said corticosteroid is from 1 to 30 mg of prednisolone, or an equivalent, equipotent amount of another corticosteroid.

18. A method for treating a Group A musculoskeletal disorder in a patient in need thereof, said method comprising administering to said patient a first dose of a first corticosteroid at a time T₁ followed by a second dose of a second corticosteroid at a time T₂, wherein said time T₁ and said time T₂ are separated by 2 to 10 hours and wherein said first dose of a corticosteroid and said second dose of a corticosteroid are administered in amounts that together are sufficient to treat said patient.

19. A method for treating a Group A musculoskeletal disorder in a patient in need thereof, said method comprising administering to said patient a pharmaceutical composition in unit dosage form comprising a first dose of a first corticosteroid formulated for immediate release and a second dose of a second corticosteroid formulated for 2 to 10 hour delayed release, wherein said first dose and said second dose are administered in amounts that together are sufficient to treat said patient.

20. A method for treating a Group A musculoskeletal disorder in a patient in need thereof, said method comprising administering to said patient a pharmaceutical composition in unit dosage form comprising a corticosteroid formulated for 2 to 10 hour sustained release, wherein said corticosteroid is from 1 to 30 mg of prednisolone, or an equivalent, equipotent amount of another corticosteroid.

21. The method of any of claims 18-20, wherein said Group A musculoskeletal disorder is selected from arthritis, ankylosing spondylitis, Behcet's disease, bursitis, dermatomyositis, fasciitis, fibromyalgia, lupus, myositis, myositis ossificans, necrotizing fasciitis, polymyalgia rheumatica, psoriatic arthritis, relapsing polychondritis, rheumatic fever, scleroderma, Sjögren's syndrome, Still's disease, and Wegener's granulomatosis.

22. A method for treating a Group B musculoskeletal disorder in a patient in need thereof, said method comprising administering to said patient a first dose of a first corticosteroid at a time T₁ followed by a second dose of a second corticosteroid at a time T₂, wherein said time T₁ and said time T₂ are separated by 2 to 10 hours and wherein said first dose of a corticosteroid and said second dose of a corticosteroid are administered in amounts that together are sufficient to treat said patient.

23. A method for treating a Group B musculoskeletal disorder in a patient in need thereof, said method comprising administering to said patient a pharmaceutical composition in unit dosage form comprising a first dose of a first corticosteroid formulated for immediate release and a second dose of a second corticosteroid formulated for 2 to 10 hour delayed release, wherein said first dose and said second dose are administered in amounts that together are sufficient to treat said patient.

24. A method for treating a Group B musculoskeletal disorder in a patient in need thereof, said method comprising administering to said patient a pharmaceutical composition in unit dosage form comprising a corticosteroid formulated for 2 to 10 hour sustained release, wherein said corticosteroid is from 1 to 30 mg of prednisolone, or an equivalent, equipotent amount of another corticosteroid.

25. The method of any of claims 22-24, wherein said Group B musculoskeletal disorder is selected from acquired hyperostosis syndrome, acromegaly, chronic fatigue syndrome, congenital hypothyroidism, dentigerous cyst, diffuse idiopathic skeletal hyperostosis, Dupuytren's contracture, eosinophilia-myalgia syndrome, Felty's syndrome, hallux valgus, Kabuki make-up syndrome, Legg-Perthes disease, Lyme disease, Melas syndrome, neurogenic arthropathy, osteitis deformans, osteochondritis, osteomalacia, osteomyelitis, osteonecrosis, osteoporosis, Paget's disease, Pierre Robin syndrome, polymyositis, postpoliomyelitis syndrome, pseudogout, Reiter disease, renal osteodystrophy, rhabdomyolysis, Sever's disease (calceneal apophysitis), spinal stenosis, synovitis, tendinopathy, tennis elbow, tenosynovitis, and Tietze's syndrome.

26. A method for treating pain in a patient in need thereof, said method comprising administering to said patient a first dose of a first corticosteroid at a time T₁ followed by a second dose of a second corticosteroid at a time T₂, wherein said time T₁ and said time T₂ are separated by 2 to 10 hours and wherein said first corticosteroid and said second corticosteroid are administered in amounts that together are sufficient to treat said patient.

27. A method for treating pain in a patient in need thereof, said method comprising administering to said patient a pharmaceutical composition in unit dosage form comprising a first dose of a first corticosteroid formulated for immediate release and a second dose of a second corticosteroid formulated for 2 to 10 hour delayed release, wherein said first dose and said second dose are administered in amounts that together are sufficient to treat said patient.

28. A method for treating pain in a patient in need thereof, said method comprising administering to said patient a pharmaceutical composition in unit dosage form comprising a corticosteroid formulated for 2 to 10 hour sustained release, wherein said corticosteroid is from 1 to 30 mg of prednisolone, or an equivalent, equipotent amount of another corticosteroid.

29. The method of any of claims 26-28, wherein said pain is inflammatory pain, neuropathic pain, or nociceptive pain.

30. A method for treating periodontal disease in a patient in need thereof, said method comprising administering to said patient a first dose of a first corticosteroid at a time T₁ followed by a second dose of a second corticosteroid at a time T₂, wherein said time T₁ and said time T₂ are separated by 2 to 10 hours and wherein said first dose and said second dose are administered in amounts that together are sufficient to treat said patient.

31. A method for treating periodontal disease in a patient in need thereof, said method comprising administering to said patient a pharmaceutical composition in unit dosage form comprising a first dose of a first corticosteroid formulated for immediate release and a second dose of a second corticosteroid formulated for 2 to 10 hour delayed release, wherein said first dose and said second dose are administered in amounts that together are sufficient to treat said patient.

32. A method for treating periodontal disease in a patient in need thereof, said method comprising administering to said patient a pharmaceutical composition in unit dosage form comprising a corticosteroid formulated for 2 to 10 hour sustained release, wherein said corticosteroid is from 1 to 30 mg of prednisolone, or an equivalent, equipotent amount of another corticosteroid.

33. The method of any of claims 30-32, wherein said periodontal disease is periodontitis or gingivitis.

34. The method of any of claims 1-33, wherein said first dose and said second dose together are equal to or less than 30 mg of prednisolone or an equivalent, equipotent amount of another corticosteroid.

35. The method of claim 34, wherein said first dose and said second dose together are equal to or less than 7.5 mg of prednisolone or an equivalent, equipotent amount of another corticosteroid.

36. The method of claim 34, wherein said first dose is from 0.25 mg to 10 mg of prednisolone or an equivalent, equipotent amount of another corticosteroid and said second dose is from 5 mg to 25 mg of prednisolone or an equivalent, equipotent amount of another corticosteroid.

37. The method of claim 34, wherein said time T₁ and said time T₂ are separated by 2 to 6 hours.

38. The method of claim 34, wherein said first dose is formulated for immediate release and said second dose is formulated for delayed release and wherein said first dose and said second dose are each formulated in unit dosage form for oral administration.

39. The method of claim 38, wherein said second dose is formulated for 2 to 8 hour delayed release.

40. The method of claim 39, wherein said first corticosteroid and said second corticosteroid are each prednisolone.

41. A kit comprising:
(i) a first composition comprising a first dose of a first corticosteroid;
(ii) a second composition comprising a second dose of a second corticosteroid; and
(iii) instructions for administering said first composition at a time T₁ followed by said second composition at a time T₂ to a patient diagnosed with or at risk of developing an immunoinflammatory disorder, to a patient for the treatment of pain or fatigue associated with a musculoskeletal disorder, to a patient for the treatment of tenderness, impairment in mobility, soft tissue swelling, or bony swelling associated with a musculoskeletal disorder, to a patient diagnosed with or at risk of developing a Group A musculoskeletal disorder, to a patient diagnosed with or at risk of developing a Group B musculoskeletal disorder, to a patient for the treatment of pain, to a patient for the treatment of periodontal disease, or to a patient diagnosed with or at risk of developing a disease or condition associated with an increased serum CRP levels, wherein said time T₁ and said time T₂ are separated by 2 to 10 hours.

42. A kit comprising:
(i) a pharmaceutical composition in unit dosage form comprising a first dose of a first corticosteroid formulated for immediate release and a second dose of a second corticosteroid formulated for 2 to 10 hour delayed release; and
(ii) instructions for administering said pharmaceutical composition to a patient diagnosed with or at risk of developing an immunoinflammatory disorder, to a patient for the treatment of pain or fatigue associated with a musculoskeletal disorder, to a patient for the treatment of tenderness, impairment in mobility, soft tissue swelling, or bony swelling associated with a musculoskeletal disorder, to a patient diagnosed with or at risk of developing a Group A musculoskeletal disorder, to a patient diagnosed with or at risk of developing a Group B musculoskeletal disorder, to a patient for the treatment of pain, to a patient for the treatment of periodontal disease, or to a patient diagnosed with or at risk of developing a disease or condition associated with an increased serum CRP levels.

43. A kit comprising:
(i) a pharmaceutical composition in unit dosage form comprising a corticosteroid formulated for 2 to 10 hour sustained release, wherein said corticosteroid is from 1 to 30 mg of prednisolone, or an equivalent, equipotent amount of another corticosteroid; and
(ii) instructions for administering said pharmaceutical composition to a patient diagnosed with or at risk of developing an immunoinflammatory disorder, to a patient for the treatment of pain or fatigue associated with a musculoskeletal disorder, to a patient for the treatment of tenderness, impairment in mobility, soft tissue swelling, or bony swelling associated with a musculoskeletal disorder, to a patient diagnosed with or at risk of developing a Group A musculoskeletal disorder, to a patient diagnosed with or at risk of developing a Group B musculoskeletal disorder, to a patient for the treatment of pain, to a patient for the treatment of periodontal disease, or to a patient diagnosed with or at risk of developing a disease or condition associated with an increased serum CRP levels.

44. The kit of any of claims 41-43, wherein said first dose and said second dose together are equal to or less than 30 mg of prednisolone or an equivalent, equipotent amount of another corticosteroid.

45. The kit of any of claims 41-43, wherein the ratio of corticosteroid in said first dose to corticosteroid in said second dose is from 1:2 to 2:1.

46. The kit of any of claims 41-43, wherein said first dose is from 0.25 mg to 10 mg of prednisolone or an equivalent, equipotent amount of another corticosteroid and said second dose is from 5 mg to 25 mg of prednisolone or an equivalent, equipotent amount of another corticosteroid.

47. The kit of claim 46, wherein said time T₁ and said time T₂ are separated by 2 to 6 hours.

48. The kit of claim 47, wherein said first dose is formulated for immediate release and said second dose is formulated for delayed release and wherein said first dose and said second dose are each formulated in unit dosage form for oral administration.

49. The kit of claim 48, wherein said first corticosteroid and said second corticosteroid are each prednisolone.

50. A pharmaceutical composition formulated for oral administration in unit dosage form comprising a first dose of a first corticosteroid formulated for immediate release and a second dose of a second corticosteroid formulated for 2 to 10 hour delayed release.

51. The pharmaceutical composition of claim 50, wherein said first dose and said second dose together are equal to or less than 30 mg of prednisolone or an equivalent, equipotent amount of another corticosteroid.

52. The pharmaceutical composition of claim 51, wherein said first dose and said second dose together are equal to or less than 7.5 mg of prednisolone or an equivalent, equipotent amount of another corticosteroid.

53. The pharmaceutical composition of claim 50, wherein the ratio of said first dose to said second dose is from 1:2 to 2:1.

54. The pharmaceutical composition of claim 50, wherein said first dose is from 0.25 mg to 10 mg of prednisolone or an equivalent, equipotent amount of another corticosteroid and said second dose is from 5 mg to 25 mg of prednisolone or an equivalent, equipotent amount of another corticosteroid.

55. The pharmaceutical composition of claim 54, wherein said first corticosteroid and said second corticosteroid are each prednisolone.

56. A pharmaceutical composition formulated for oral administration in unit dosage form comprising a corticosteroid formulated for 2 to 10 hour sustained release, wherein said corticosteroid is from 1 to 30 mg of prednisolone, or an equivalent, equipotent amount of another corticosteroid.
